SEQUENCE LISTING (3 sequences)

SEQ ID NO: 1 - RNA, 1523 nt, Enterococcus faecium
acgcgggcggcgtgcctaatacatgcaagtcgtacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgatgggggtgaagtcgtaacaaggtagccgtatcggaaggtgcggctggatcccc

SEQ ID NO: 2 - DNA, 17 bp, Artificial Sequence
  universal forward primer 785F
ggttagataccctggta

SEQ ID NO: 3 - DNA, 20 bp, Artificial Sequence
  universal reverse primer  907R
ccgtcaattcmtttragttt

(19) United States
(12) Patent Application Publication
(10) Pub. No.: US 2021/0329943 A1
(43) Pub. Date: Oct. 28, 2021

(54) FERMENTED SOYBEAN MEAL AND PREPARATION METHOD THEREFOR

(71) Applicant: FEEDUP CO., LTD., Nonsan-si (KR)

(72) Inventors: Jong Hwa LEE, Sejong (KR); Hyuck Gee LEE, Icheon-si (KR)

(21) Appl. No.: 17/259,631

(22) PCT Filed: Feb. 1, 2019
(86) PCT No.: PCT/KR2019/001462

§ 371 Date: Jan. 12, 2021

(30) Foreign Application Priority Data
Jul. 17, 2018 (KR) 10-2018-0083163
Aug. 30, 2018 (KR) 10-2018-0102926
Jan. 24, 2019 (KR) 10-2019-0009489
Jan. 31, 2019 (KR) 10-2019-0013058

(51) Int. Cl. A23K 10/12; C12N 1/20; A23K 20/163; A23K 20/147; A23K 50/80; A23K 50/30; A23K 50/75

(52) U.S. Cl. CPC A23K 10/12; C12N 1/205; A23K 20/163; C12R 2001/46; A23K 50/80; A23K 50/30; A23K 50/75; A23K 20/147

(57) ABSTRACT

The present invention relates to a method for preparing fermented soybean meal, and more specifically, relates to a method for preparing fermented soybean meal using a facultative anaerobic soybean meal fermenting microorganism, fermented soybean meal prepared therefrom, and a feed composition comprising the same. In addition, the present invention relates to a novel Enterococcus sp. strain, and more specifically, relates to an Enterococcus faecium strain.

### TECHNICAL FIELD

The present invention relates to fermented soybean meal, a method for preparing fermented soybean meal, and a feed composition comprising fermented soybean meal. In addition, the present invention relates to a novel Enterococcus sp. strain, a method for preparing fermented soybean meal using the strain, fermented soybean meal prepared therefrom, and a feed composition comprising the same.

### BACKGROUND ART

Defatted soybean meal accounts for 60% of the world's vegetable protein feed raw material on a production basis. As recently the negative outlook for animal protein sources has been increased, because of the outbreak of bovine spongiform encephalopathy, etc., it is expected that the demand for defatted soybean meal will be continuously increased in the future.

To enhance the productivity of livestock, an activator, an antibiotic, an antimicrobial agent, a growth hormone drug, etc. can be used, but because of problems of the antibiotic, etc. of increasing resistance of pathogenic bacteria or being remained in livestock products, etc., the use of microorganism preparations such as probiotic has been recently increased. Such microorganism preparations can be used for purposes of facilitating enzyme secretion in the animal body, improving the digestion rate of indigestible fibrin, and reducing anti-nutritional factors, etc.

Among soybean components, anti-nutritional factors such as trypsin inhibitor are weak in heat and thus most of them are inactivated in the process of production of soybean meal, but for indigestible oligosaccharides such as raffinose and stachyose which are strong in heat, a method of decomposition using microbial fermentation or an enzyme is usually used.

A solid substrate fermentation method is usually used for fermenting soybean meal around the world, and an aerobic fermentation method, which uses an aerobic microorganism that usually uses fungi represented by Aspergillus sp. and bacteria represented by Bacillus sp. as a microorganism and supplies oxygen forcibly, becomes mainstream. This aerobic solid substrate fermentation method has problems that it necessarily requires oxygen supply and therefore the possibility of contamination is high and considerable level of facility investment such as a koji making machine, etc. is needed, and the factory scale-up is not easy for increasing output, etc. Accordingly, the solid substrate fermentation method using anaerobic bacteria is stated to be an alternative which can overcome such problems of the aerobic process.

The anaerobic bacterium, lactic acid bacteria convert one of the representative anti-nutritional factors, indigestible oligosaccharides into beneficial substances such as lactic acid, etc., and are useful for livestock as amino acid metabolism is active and also make various biologically active substances. In particular, since the lactic acid secreted in the process of fermentation of lactic acid bacteria can inhibit proliferation of other microorganisms, it has an advantage of minimizing contamination. However, the conventional anaerobic soybean meal fermentation process which usually uses lactic acid bacteria of Lactobacillus sp. has a disadvantage of taking a lot of time and requiring excessive equipment for securing the anaerobic condition, since the lactic acid bacteria have slower speed than aerobic bacteria in the growth and development, and fermentation.

### DISCLOSURE

### Technical Problem

The present invention provides a method for preparation of fermented soybean meal using soybean meal comprising saccharides and some protein in soybean meal, and a microorganism fermenting soybean meal.

Another purpose of the present invention is to provide a method for controlling the crude protein content of fermented soybean meal, by preparing soybean meal extract solution and residual soybean meal using an extraction solvent and using them as a fermentation raw material.

In addition, the present invention provides fermented soybean meal having an appropriate content of crude protein according to the object livestock species and growth section of livestock, and a feed composition comprising the fermented soybean meal.

Moreover, the present invention prepares soybean meal extract solution comprising saccharides and some protein in soybean meal, and selects a novel strain showing high specific growth rate when using it as a medium, thereby creating an extraction and fermentation process of soybean meal based on the novel strain.

### Technical Solution

Soybean meal is used as feed, etc. by itself, but it may be prepared as soybean meal in which raw soybean meal is treated by using an enzyme or microorganism, or residual soybean in which unnecessary components were removed with an extraction solvent, for purposes of facilitating enzyme secretion in animal body, improving the digestion rate of indigestible fibrin and reducing anti-nutritional factors, etc., through a fermentation process. The preparation of a fermentation raw material using an extraction solvent is to remove anti-nutritional factors of soybean meal and to remove it by selectively extracting components except for protein to concentrate the crude protein content. The anti-nutritional factors include trypsin inhibitor, indigestible oligosaccharides such as raffinose and stachyose, etc.

One example of the present invention relates to a method for preparing fermented soybean meal by preparing soybean extract solution and residual soybean meal by using an extraction solvent, and fermenting by using them as fermentation raw materials with a microorganism fermenting soybean meal. The soybean meal fermenting microorganism may be Enterococcus faecium SLB130.

In addition, the residual soybean meal obtained by extracting the raw soybean meal with an extraction solvent has the high crude protein content, and it can be used by adjusting the crude protein content to be suitable for purposes of use. Accordingly, the crude protein content of the soybean meal fermented product may be controlled by lowering the crude protein content by using the residual soybean meal obtained after extraction or mixing raw soybean meal to the residual soybean meal. For example, solid culturing only the residual soybean meal as the fermentation raw material is appropriate for production of a relatively high concentration of crude protein product, and it can be used as an alternative of fishmeal used for feed for fish. The feed composition may be used as a feed composition for adult livestock, piglets or hatchery fish according to the crude protein content.

In addition, the required level of anti-nutritional factors and the crude protein content depending on livestock are different respectively, and to prepare fermented soybean meal in quality required for each object livestock, it is important to achieve the appropriate anti-nutritional factor level and the crude protein content.

Accordingly, another example of the present invention relates to a method for controlling the mixing ratio of residual soybean meal and raw soybean meal in consideration of the level of anti-nutritional factors and the crude protein content required in used livestock and preparing fermented soybean meal in quality required for each object livestock.

For example, for fermented soybean meal which is appropriate for hatchery fish such as shrimp, abalone or salmon, etc., fermented soybean meal may be prepared by using residual soybean meal as a fermentation raw material. In other words, solid culturing residual soybean meal after extraction is proper for production of a high concentration of crude protein product, and it may be used as an alternative of fishmeal for hatchery fish. Specifically, it is preferable that the content of anti-nutritional factors of fermented soybean meal is less than 0.03% in case of indigestible oligosaccharides, and is low as 200 ppm in case of beta-conglycinin, and is high as 58% or more in case of the crude protein content. Specifically, in the fermented soybean meal which is appropriates for fish or hatchery fish, the crude protein content may be 53 to 65% (w/w), higher than 53 to 65% (w/w), 54 to 65% (w/w), 55 to 65% (w/w), 56 to 65% (w/w), higher than 56 to 65% (w/w), 57 to 65% (w/w), 58 to 65% (w/w), 59 to 65% (w/w), or 60 to 65% (w/w).

For example, as the adult livestock such as mother pig, growing pig or domestic cow, etc. has well developed digestive organs, it is no matter that the concentration of anti-nutritional factors in fermented soybean meal is relatively high, and it is no matter that the absorption rate of amino acids is higher than general soybean meal and the crude protein content is 50% or more. Thus, the fermented soybean meal for adult livestock may be prepared by using raw soybean meal. Specifically, in the fermented soybean meal which is appropriate for adult livestock such as mother pig, growing pig or domestic cow, etc., the crude protein content may be 48 to 53% (w/w), higher than 48 to lower than 51% (w/w), higher than 48 to 50% (w/w), 49 to 53% (w/w), 49 to 51% (w/w), 49 to lower than 51% (w/w), 49 to 50% (w/w), higher than 49 to lower than 53% (w/w), higher than 49 to 51%% (w/w), or higher than 49 to lower than 51% (w/w).

For example, in case of young livestock such as piglet, young chick, etc., the effect by anti-nutritional factors is small than the case of hatchery fish, but it is affected considerably largely by anti-nutritional factors than adult livestock. Thus, in the fermented soybean meal for young livestock, the content of anti-nutritional factors is less than 0.05% in case of indigestible oligosaccharides, and less than 500 ppm in case of beta-conglycinin. Thus, the fermented soybean meal for young livestock may be prepared by mixing the residual soybean meal and raw soybean meal, and the mixing ratio thereof may be 1:1 to 1:1.4. Specifically, in the fermented soybean meal for young livestock such as piglet, young chick, etc., the crude protein content may be higher than 50 to lower than 60% (w/w), higher than 50 to 59% (w/w), higher than 50 to 58% (w/w), higher than 50 to 57% (w/w), higher than 50 to 56% (w/w), higher than 50 to lower than 56% (w/w), higher than 50 to 55% (w/w), higher than 50 to 54% (w/w), higher than 50 to 53% (w/w), 51 to lower than 60% (w/w), 51 to 59% (w/w), 51 to 58% (w/w), 51 to 57% (w/w), 51 to 56% (w/w), 51 to 55% (w/w), 51 to lower than 55% (w/w), 51 to 55% (w/w), 51 to 54% (w/w), 51 to 53% (w/w), higher than 51 to lower than 60% (w/w), higher than 51 to 59% (w/w), higher than 51 to 58% (w/w), higher than 51 to 57% (w/w), higher than 51 to 56% (w/w), higher than 51 to lower than 56% (w/w), higher than 51 to 55% (w/w), higher than 51 to 54% (w/w), higher than 51 to 53% (w/w), 52 to lower than 60% (w/w), 52 to 59% (w/w), 52 to 58% (w/w), 52 to 57% (w/w), 52 to 56% (w/w), 52 to lower than 56% (w/w), 52 to 55% (w/w), 52 to 54% (w/w), 52 to 53% (w/w), 53 to lower than 60% (w/w), 53 to 59% (w/w), 53 to 58% (w/w), 53 to 57% (w/w), 53 to 56% (w/w), 53 to lower than 56% (w/w), 53 to 55% (w/w), or 53 to 54% (w/w).

Accordingly, one example of the present invention provides a method for preparing a soybean meal fermented product by fermenting one or more kinds of fermentation raw materials selected from the group consisting of raw soybean meal and residual soybean meal with a a microorganism fermenting soybean meal, or a method for controlling the crude protein content of a soybean meal fermented product. The soybean meal fermenting microorganism may be Enterococcus faecium SLB130 strain.

The present invention provides a method for producing fermented soybean meal having high cost competitiveness in the animal feed market by an anaerobic fermentation process technology which can produce fermented soybean meal with relatively simple process equipment and low cost, as an alternative to the conventional aerobic fermentation process which requires a lot of facility and operating costs, by using facultative anaerobic lactic acid bacteria as the soybean meal fermenting microorganism. The fermentation process in the present invention may be stably operated, since the fermentation time is considerably short and the possibility of contamination is low. The facultative anaerobic lactic acid bacteria may be Enterococcus faecium SLB130 strain.

Since the present invention prepares soybean meal extract solution and residual soybean meal using an extraction solvent and prepares a soybean meal fermented product by performing fermentation by using them as fermentation raw materials, the indigestible oligosaccharides that significantly reduce the feed efficiency of soybean meal by disturbing the digestive absorption of livestock are excellently removed in the process of the present invention. In addition, fermented soybean meal segmented according to livestock species and objects may be produced by using the extraction process.

The present invention uses the discarded soybean meal extract solution as lactic acid bacteria culture medium, and thereby the cost can be reduced, and also an environment-friendly method without process waste is provided.

In addition, the present invention suggests a technological basis which can create added values by not only using the soybean meal extract solution as a lactic acid bacterium culturing medium, but also obtaining products such as lactic acid, etc. by culturing a microorganism, and can produce various fermented products having an industrial value through fermentation of soybean meal extract solution. The lactic acid required for preparation of PLA is mostly produced by fermentation by using lactic acid bacteria, and when the extract solution and lactic acid bacteria in the present invention are used, an economical lactic acid preparation process can be made.

In the present invention, it was confirmed that the growth and development of the lactic acid bacterium of Enterococcus sp. selected in the present invention in the soybean meal extract solution was significantly excellent compared to other lactic acid bacteria or Bacillus subtilis, etc. (Example 3). In addition, this lactic acid bacterium has been well known as a lactic acid bacterium kind which raises the digestive absorption rate by being added to feed for livestock or animals as pets, and marine products. The Enterococcus faecium SLB130 strain has advantages of not only active growth and development in soybean meal extract solution but also easy formulation.

In the present invention, through a simple and economical process of inoculating and mixing a microorganism fermenting soybean meal to a fermentation raw material and fixing, a necessary and sufficient fermentation effect for preparation of fermented soybean meal can be obtained. However, for this result, a sufficiently large amount of inoculation of the soybean meal fermenting microorganism is required, and this can be solved by using the high concentration of culture solution prepared by the soybean meal extract solution.

In the present invention, a high concentration culture method using soybean meal extract solution as a medium has been developed to produce a large amount of lactic acid bacteria inoculum. For this, a strain showing a high level of growth and development in the soybean meal extract solution prepared by the above method at the temperature of 40° C. or more was investigated. Specifically, the growth and development rate of microorganisms at 40° C. or more was measured by using soybean extract solution in which nothing was added as a medium. This is because the environment where the microorganism experiences is close to liquid state in which aqueous organic matters of soybean meal are abundantly dissolved, as large amount of water is added for activities of the microorganism in the actual solid fermentation of soybean meal, and in addition, it is to use the soybean meal extract solution to obtain a large amount of lactic acid bacteria inoculum. As the medium culturing the strain, MRS medium was used as a selective medium.

The present invention relates to Enterococcus faecium SLB130 strain.

The strain may be deposited as the accession number of KCTC13566BP.

According to other one example of the present invention, it relates to a method for preparation of fermented soybean meal comprising a step of inoculating Enterococcus faecium SLB130 strain to soybean meal.

According to other one example of the present invention, it is to provide a method for preparation of a soybean meal fermented product by solid fermentation, which is characterized by comprising a step of inoculating Enterococcus faecium SLB130 strain to soybean meal.

According to other one example of the present invention, it is to provide a method for preparation of a soybean meal fermented product comprising a step of preparing one or more kinds of fermentation raw materials selected from the group consisting of raw soybean meal and residual soybean meal obtained by extracting the raw soybean meal with an extraction solvent; a step of seed culturing which inoculates Enterococcus faecium SLB130 strain to soybean meal extract solution obtained by extracting the raw soybean meal with an extraction solvent and seed cultures it; and a step of solid culturing by inoculating the seed to the fermentation raw materials.

The raw material medium of the seed culturing or solid culturing may be (1) raw soybean meal, (2) soybean meal extract solution, (3) residual soybean meal after extraction, or (4) a mixture of raw soybean meal and residual soybean meal after extraction.

Other one example of the present invention is to extract certain components from soybean meal and culture lactic acid bacteria by using the extract, and then inoculates the cultured lactic acid bacteria to soybean meal to solid culture it. In addition, it may comprise a step of recycling the lactic acid bacteria culture solution.

Hereinafter, the present invention will be described in more detail.

In the present invention, “soybean meal” may be one or more kinds selected from the group consisting of (1) raw soybean meal, (2) soybean extract solution, and (3) residual soybean meal after extraction.

In the present invention, the term “raw soybean meal” means soybean meal before extracting soybean meal with a solvent. The raw soybean meal contains a lot of protein and thus it is a major protein source of livestock feed. The soybean meal may collectively refer to all of common defatted soybean meal and soybean meal, but not limited thereto.

In the present invention, the term “soybean meal extract” means soybean extract obtained by extracting raw soybean meal with a solvent, and the soybean meal extract includes “soybean extract solution” and “residual soybean meal” remained after removing it.

By performing the solvent extraction process, anti-nutritional factors and sugars comprised in raw soybean meal may be reduced or removed. In the raw soybean meal, polysaccharides such as stachyose, raffinose, etc. and various carbohydrates are comprised, and these are mostly aqueous and include anti-nutritional factors (hereinafter, ANF) which disturb digestion of livestock. In addition, the crude protein content in raw soybean meal may be concentrated through the extraction process, and thereby the crude protein content in residual soybean meal and fermented soybean meal using it may be increased.

In the present invention, “anti-nutritional factor” may include all the substances which disturb digestion of livestock, and may include all negative substances for digestion comprised in soybean meal or fermented soybean meal. For example, the anti-nutritional factor may be one or more kinds selected from the group consisting of trypsin inhibitor, beta-conglycininin, indigestible oligosaccharides, hemagglutinin (lectin), saponin and tannin.

To remove anti-nutritional factors of soybean meal and concentrate the crude protein content, after removing components except for protein, enzyme treatment or fermentation should be conducted. For example, when indigestible oligosaccharides such as raffinose and stachyose that are anti-nutritional factors present in soybean meal are first extracted and removed, the anti-nutritional factors are removed and at the same time, the crude protein content of residual soybean meal remained after extraction is increased. In other words, through the extraction process of raw soybean meal, the crude protein content in residual soybean meal and fermented soybean meal using it can be controlled.

The extraction of soybean meal of the present invention uses a method of extracting non-protein components in soybean meal effectively by using acidic water as a solvent. It is a principle that protein is precipitated and therefore is not extracted, and aqueous oligosaccharides and carbohydrates that have no effect on pH escape, when extraction is carried out by using weakly acidic water having pH of less than 4.5 as a solvent, by using that the isoelectric point (hereinafter, pI) of most protein in soybean meal is 4.5.

To ferment soybean meal in quantity, a great amount of inoculum is needed. In particular, it is especially true in case of solid fermentation which makes it difficult to control the fermentation environment. Generally, since it is not difficult to make homogeneous environment in liquid fermentation, temperature and pH control, oxygen supply (or oxygen block), etc. are easy, and therefore a great deal of inoculum is not needed. Commonly, the inoculum size of about from 10⁶ to 10⁷ cfu (colony forming unit) per ml, or 1-5% in volume is used.

In particular, to ferment soybean meal by using lactic acid bacteria, there are various advantages in using a sufficiently large amount of inoculum. As the growth and development rate of lactic acid bacteria which are grown and developed anaerobically (or facultative anaerobically) is slow than aerobic bacteria or yeast, fungus, etc., a larger amount of inoculum is needed to effectively ferment soybean meal.

When selecting a fermenting microorganism, it is common to select a microorganism which has an excellent decomposition ability by targeting the most or major one among organic components possessed by the object. Thus, when investigating a microorganism for fermenting soybean meal, it is a common method to find a microorganism which produces an enzyme that decomposes sugars largely composed in soybean meal, in particular, raffinose and stachyose that are indigestible oligosaccharides, and produces an enzyme that decomposes soybean meal protein a lot. This is because representative anti-nutritional factors of soybean, trypsin inhibitor (TI) and beta-conglycininin, etc. consist of protein, and it is good to decompose protein at a level of small size of protein or peptide so that livestock digests soybean protein having relatively large molecular weight smoothly. In addition, the secondary condition is a microorganism which can cause fermentation by adapting to soybean meal solid fermentation conditions, for example, moisture content, temperature, pH, etc. General bacteria present in the common natural environment not enterobacteria have many beneficial points for fermentation in particular, as the optimum temperature of growth and development is higher, since there are many bacteria that cannot grow at 40° C. or more. Moreover, as the body temperature is about 41° C. in case of chicken or cow, it may be a characteristic of lactic acid bacteria which is advantageous for survival and proliferation in the actual intestine of livestock.

In the present invention, fermented soybean meal is prepared by obtaining extracts from soybean meal and selecting and culturing lactic acid bacteria that grow well under the anaerobic condition to the extracts, and then anaerobically fermenting soybean meal with the cultured lactic acid bacteria. Hereinafter, soybean meal is a common name for both common defatted soybean meal and soybean meal.

Soybean meal contains a large amount of protein and thus is a major protein source of livestock feed. A soy protein concentrate (hereinafter, SCP) that anti-nutritional factor components are removed in soybean meal and the protein portion is increased is a high-class product used for feed of weaning stage piglets, etc., a mixture of ethyl alcohol and water is used as an extraction solvent. However, there is a disadvantage in the process that the alcohol used as a solvent requires expensive recovery equipment. In addition, remained alcohol components may inhibit the growth and development of microorganisms. In the present invention, non-protein components of soybean meal are extracted by using acidic water as a solvent instead of alcohol having such a disadvantage.

Since the isoelectric point (hereinafter, pI) of protein in soybean meal is about 4.5, when extracting by using weakly acidic water having pH of less than 4.5 as a solvent, protein is precipitated and therefore is not extract, and aqueous oligosaccharides and carbohydrates which have no effect on pH are extracted. To ferment soybean meal in quantity, a large amount of inoculum is needed. Since it is difficult for solid fermentation to create homogeneous environment, different from liquid fermentation, it is to maintain sterilization and fermentation conditions. Thus, further more inoculum than liquid fermentation should be used, and this necessarily increases process cost.

Generally, in the lactic acid bacteria fermentation process, lactic acid is secreted, and this shows an effect of decreasing pH of a medium, and finally plays a role of blocking proliferation of other contaminants. On the other hand, as the growth and development rate of lactic acid bacteria which grow and develop anaerobically (or facultative anaerobically) is slow than aerobic bacteria or yeast, fungus, etc., a larger amount of inoculum is needed to effectively ferment soybean meal by using lactic acid bacteria. General bacteria present in the common natural environment not enterobacteria have many beneficial points for fermentation in particular, as the optimum temperature of growth and development is higher, since there are many bacteria that cannot grow at 40° C. or more. Moreover, as the body temperature is about 41° C. in case of chicken or cow, it may be a characteristic of lactic acid bacteria which is advantageous for survival and proliferation in the actual intestine of livestock.

In the present invention, to produce a great amount of lactic acid bacteria inoculum, a high concentration culture method which uses soybean meal extract solution as a medium has been developed. For this, first, lactic acid bacteria showing high level of growth and development in the soybean meal extract solution prepared by the method at the temperature of 40° C. or more have been investigated.

In the present invention, the growth and development rate of microorganisms was measured at 40° C. or more by using soybean meal extract solution in which nothing was added as a liquid medium. This is because the environment where microorganisms experience is close to liquid in which aqueous organic matters of soybean meal are dissolved abundantly, as a great amount of water is added for activities of microorganism in the actual solid fermentation of soybean meal, and in addition, this is for using the soybean meal extract solution to obtain a large quantity of lactic acid bacteria inoculum. As the medium culturing lactic acid bacteria, MRS medium was used as a selective medium.

The strain of lactic acid bacteria of the present invention effectively decomposes and uses soybean meal organic matters such as oligosaccharides, carbohydrates, protein, etc., and in particular, sucrose, stachyose, raffinose, etc. which common microorganisms cannot use well are used as a carbon source. Like this, that microorganisms can be cultured by using extracts in which oligosaccharides that decrease the performance of soybean meal as an anti-nutritional factor are removed as a medium can not only reduce cost for preparation of inoculum for fermentation of soybean meal but also produce new values through additional production of industrial lactic acid bacteria powder, or lactic acid, amino acid, etc.

In the present invention, a continuous culture method has been invented to culture the selected lactic acid bacteria at a high concentration in soybean meal extract solution. The continuous culture method can obtain culture products continuously by adding a new medium to a smaller culture tank than a batch type culture method.

The continuous culture method in the present invention does not use a base such as sodium hydroxide or a lactic acid neutralizing agent such as calcium carbonate on the basis that the specific growth rate of the selected lactic acid bacteria is significantly high, and makes soybean meal extract solution added automatically so that certain pH is maintained. This method can increase the recyclability of resources and can simplify the process in the aspect that no additional additives are used.

In order to remove anti-nutritional factors of soybean meal and concentrate the crude protein content, after removing components except for protein by selective extraction, enzyme treatment or fermentation should be carried out. This is because most of soybean meal anti-nutritional factors have a protein structure. Recently, such a purpose is achieved through fermentation, and Bacillus sp. bacteria or Aspergillus sp. fungus or Saccharomyces (beer yeast/baker's yeast) sp. yeast, etc. are usually used.

When fermenting by using such aerobic microorganisms, the initial investment cost, operating cost, cost price, etc. are increased, and most importantly, the enlargement of production scale is not easy. In other words, in the aerobic process, a great deal of effort is required of supply a uniform and sufficient amount of oxygen, and because of this, the thickness to be piled up is limited, and as a result, a lot of space is required in the facility, and the use of resources such as water vapor, water, air, heat, etc. is not effective relatively.

In contrast, the anaerobic process does not require the supply of oxygen, so it is a great advantage that fermentation conditions can be maintained by stacking without expensive/high cost of a koji making machine. Since there is heat generated during the fermentation process, there is no need for additional facilities and energy to maintain the fermentation temperature. In addition, when lactic acid bacteria, which are representative anaerobic microorganisms, are used, there is a great advantage that lactic acid produced in the fermentation process decreases pH of fermented soybean meal and thereby the contamination of various bacteria can be prevented.

However, the anaerobic fermentation using lactic acid bacteria generally has a big disadvantage of slow rate. To overcome such a disadvantage, in the present invention, a lactic acid bacterium having the growth and development rate in the fermentation process of soybean meal is fast is selected (SLB130).

Lactic acid bacteria have excellent usability for soybean meal oligosaccharides, whereas there is a disadvantage of relatively low activity of protease, but it is shown that the lactic acid bacterium in the present invention has an excellent protein decomposition ability and therefore has sufficient competitiveness. As the result of identifying the lactic acid bacterium of the present invention, it is identified as faecium of Enterococcus sp. (Enterococcus faecium) strain.

In the present invention, through a simple and economical process of fixing for 10 to 24 hours after inoculating and mixing a lactic acid bacterium to the extracted soybean meal, a necessary and sufficient fermentation effect required for preparation of fermented soybean meal can be obtained. However, a sufficiently large quantity of inoculum is needed for this result, but it could be solved by using a high concentration of lactic acid bacteria culture solution prepared through the extract solution as above.

The extract solution generated in the extraction process of the present invention contains a large amount of oligosaccharides and a small amount of protein, and therefore it has the possibility to be used as a culture medium of microorganisms. Since it contains particularly raffinose, stachyose, etc. abundantly, when this is used for culture or fermentation of lactic acid bacteria which can use this well, resources that may be discarded can be recycled.

The lactic acid bacterium of Enterococcus sp. selected in the present invention is well known as lactic acid bacteria which is added to feed for livestock or animals for pets and marine products, etc. and increases the digestive absorption rate, and it is well known as an inoculation microorganism of silage or forage. Thus, surplus microbial cells except for inoculation microorganism cells required for the process of fermentation of soybean meal is formulated, and thereby it can be produced as powder for produce industrial microorganism formulations for such a purpose. Since the growth and development of lactic acid bacteria are slow and formulation for maintaining a live state for a long time is difficult, industrial lactic acid bacteria formulations are generally expensive. The lactic acid bacterium in the present invention may create considerable added values when used, as not only the growth and development in soybean meal extract solution is active, but also formulation is not difficult.

On the other hand, as the interest on the environmental contamination has been recently increased, bio-degradable plastics have received attention, and among them, a lactic acid polymer, PLA (Polylactic Acid) is representative. Lactic acid required for preparation of PLA is mostly produced by fermentation using lactic acid bacteria, and an economical lactic acid preparation process may be made when using the extract solution and lactic acid bacterium in the present invention.

The method for preparation of fermented soybean meal according to one example of the present invention comprises the following steps:

a step of obtaining soybean meal extract solution and residual soybean meal, obtained by extracting raw soybean meal with an extraction solvent, and

a step of solid culturing by inoculating a soybean meal fermenting microorganism to a fermentation raw material comprising one or more kinds selected form the group consisting of raw soybean meal and residual soybean meal.

The method for preparation of fermented soybean meal according to one example of the present invention will be described in detail by each step.

First, the method for preparation of fermented soybean meal according to one example of the present invention comprises a process of obtaining soybean meal extract solution and residual soybean meal, obtained by extracting raw soybean meal with an extraction solvent. Specifically, it may be performed by a process of extracting raw soybean meal with an extraction solvent and treating the extract by a solid-liquid separation process to obtain soybean meal extract solution and residual soybean meal.

The solvent used for extracting raw soybean meal in the present invention may be water, a lower alcohol of 1 to 6 carbon atoms, or a mixed solvent thereof (alcohol aqueous solution), and the lower alcohol of 1 to 6 carbon atoms may include one or more kinds of ethanol, methanol, propanol, butanol, etc. The extraction solvent may be controlled to an appropriate pH condition by using a pH regulator such as acid or base. For example, the example of the pH regulator may include hydrochloric acid, acetic acid, calcium phosphate, sodium hydroxide, citric acid, etc. The pH condition of the extraction solvent may be pH 7.5 or less, pH 7 or less, pH 6 or less, pH 5 or less, pH 4.5 or less, less than pH 4.5, pH 4.4 or less, pH 4.3 or less, pH 4.2 or less, pH 4.1 or less, pH 4 or less, pH 3.5 or less, pH 3 or less, or pH 2 or less, and for example, may be pH 2 to 7.5, pH 2 to 7, pH 2 to 6.5, pH 2 to 6, pH 2 to 5.5, pH 2 to 5, pH 2 to less than 4.5, pH 2 to 4.4, pH 2 to 4.3, pH 2 to 4.2, pH 2 to 4.1, pH 2 to 4, pH 2 to 3.5, pH 2 to 3, pH 2 to 2.5, pH 2.5 to 7.5, pH 2.5 to 7, pH 2.5 to 6.5, pH 2.5 to 6, pH 2.5 to 5.5, pH 2.5 to 5, pH 2.5 to less than 4.5, pH 2.5 to 4.4, pH 2.5 to 4.3, pH 2.5 to 4.2, pH 2.5 to 4.1, pH 2.5 to 4, pH 2.5 to 3.5, pH 2.5 to 3, pH 3 to 7.5, pH 3 to 7, pH 3 to 6.5, pH 3 to 6, pH 3 to 5.5, pH 3 to 5, pH 3 to less than 4.5, pH 3 to 4.4, pH 3 to 4.3, pH 3 to 4.2, pH 3 to 4.1, pH 3 to 4, pH 3 to 3.5, pH 3.5 to 7.5, pH 3.5 to 7, pH 3.5 to 6.5, pH 3.5 to 6, pH 3.5 to 5.5, pH 3.5 to 5, pH 3.5 to less than 4.5, pH 3.5 to 4.4, pH 3.5 to 4.3, pH 3.5 to 4.2, pH 3.5 to 4.1, pH 3.5 to 4, pH 4 to 7.5, pH 4 to 7, pH 4 to 6.5, pH 4 to 6, pH 4 to 5.5, pH 4 to 5, pH 4 to less than 4.5, pH 4 to 4.4, pH 4 to 4.3, pH 4 to 4.2, or pH 4 to 4.1, and preferably, may be pH 2 to 4.5.

Preferably, the extraction solvent may be water, and the pH condition may be pH 2 to less than 4.5, pH 2.5 to less than 4.5, pH 3 to less than 4.5, pH 3.5 to less than 4.5, or pH 4 to less than 4.5, and preferably may be pH 2 to less than 4.5. When alcohol is used as an extraction solvent, the alcohol recovery equipment is required and there is a side effect that remained alcohol components inhibit the growth and development of microorganisms, but when water or hydrochloric acid aqueous solution is used as an extraction solvent, there are no additional recovery equipment and negative effects on the growth and development of microorganisms, and thus it is more preferable.

The temperature of the extraction solvent may be adjusted to an appropriate temperature in consideration of soybean meal extraction efficiency, and in addition, when a fermenting microorganism is inoculated to soybean meal extraction solution after the extraction step, the temperature of the extraction solvent can be controlled in consideration of the optimum growth and development conditions of the fermenting microorganism, and thus it is preferable to set the temperature of the extraction solvent in consideration of the extraction efficiency and the following process conditions.

For example, in case of considering the extraction efficiency of soybean meal, when water is used as the extraction solvent, the temperature of the extraction solvent may be 0 to 60° C., 0 to 55° C., 0 to 50° C., 0 to less than 40° C., 0 to 35° C., 0 to 30° C., 0 to 25° C., 0 to 20° C., 0 to 15° C., 0 to 10° C., 0 to 5° C., 5 to 60° C., 5 to 55° C., 5 to 50° C., 5 to less than 40° C., 5 to 35° C., 5 to 30° C., 5 to 25° C., 5 to 20° C., 5 to 15° C., 5 to 10° C., 10 to 60° C., 10 to 55° C., 10 to 50° C., 10 to less than 40° C., 10 to 35° C., 10 to 30° C., 10 to 25° C., 10 to 20° C., 10 to 15° C., 15 to 60° C., 15 to 55° C., 15 to 50° C., 15 to less than 40° C., 15 to 35° C., 15 to 30° C., 15 to 25° C., 15 to 20° C., 20 to 60° C., 20 to 55° C., 20 to 50° C., 20 to less than 40° C., 20 to 35° C., 20 to 30° C., 20 to 25° C., 25 to 60° C., 25 to 55° C., 25 to 50° C., 25 to less than 40° C., 25 to 35° C., 25 to 30° C., 30 to 60° C., 30 to 55° C., 30 to 50° C., 30 to less than 40° C., 30 to 35° C., 35 to 60° C., 35 to 55° C., 35 to 50° C., or 35 to less than 40° C., and preferably may be 20 to less than 40° C. When alcohol or alcohol aqueous solution is used as the extraction solvent, the temperature of the extraction solvent may be raised to the temperature of 50 to 70° C. to use. In case of ethanol, since the sugar extraction efficiency in soybean meal is low when used at a room temperature, it is preferable to use it by heating to a higher temperature than a room temperature.

Otherwise, in case of considering the optimum growth and development conditions of a fermenting microorganism, it is preferable that the temperature of extracted soybean meal extract solution and the optimum growth and development temperature of a soybean meal fermenting microorganism to be inoculated to it are similar, and therefore, when Enterococcus faecium SLB130 having the optimum growth and development temperature is 40 to 45° C. is used, the temperature of the extraction solvent may be adjusted to the temperature of 20 to 60° C., or 40 to 60° C. so that the temperature of soybean meal extract solution is 40 to 45° C.

In the present invention, the extraction solvent of raw soybean meal may be extracted by mixing the extraction solvent at a weight ratio of 1:1 to 1:10, 1:1 to 1:9.5, 1:1 to 1:9, 1:1 to 1:8.5, 1:1 to 1:8, 1:1 to 1:7.5, 1:1 to 1:7, 1:1 to 1:6.5, 1:1 to 1:6, 1:1 to 1:5.5, 1:1 to 1:5, 1:1 to lower than 1:5, 1:1 to 1:4.9, 1:1 to 1:4.8, 1:1 to 1:4.7, 1:1 to 1:4.6, 1:1 to 1:4.5, 1:1 to 1:4.4, 1:1 to 1:4.3, 1:1 to 1:4.2, 1:1 to 1:4.1, 1:1 to 1:4 or less, higher than 1:2.5 to lower than 1:5, higher than 1:2.5 to 1:4.9, higher than 1:2.5 to 1:4.8, higher than 1:2.5 to 1:4.7, higher than 1:2.5 to 1:4.6, higher than 1:2.5 to 1:4.5, higher than 1:2.5 to 1:4.4, higher than 1:2.5 to 1:4.3, higher than 1:2.5 to 1:4.2, higher than 1:2.5 to 1:4.1, higher than 1:2.5 to 1:4 or less, 1:3 to lower than 1:5, 1:3 to 1:4.9, 1:3 to 1:4.8, 1:3 to 1:4.7, 1:3 to 1:4.6, 1:3 to 1:4.5, 1:3 to 1:4.4, 1:3 to 1:4.3, 1:3 to 1:4.2, 1:3 to 1:4.1, 1:3 to 1:4 or less, higher than 1:3 to lower than 1:5, higher than 1:3 to 1:4.9, higher than 1:3 to 1:4.8, higher than 1:3 to 1:4.7, higher than 1:3 to 1:4.6, higher than 1:3 to 1:4.5, higher than 1:3 to 1:4.4, higher than 1:3 to 1:4.3, higher than 1:3 to 1:4.2, higher than 1:3 to 1:4.1, higher than 1:3 to 1:4 or less, 1:3.5 to lower than 1:5, 1:3.5 to 1:4.9, 1:3.5 to 1:4.8, 1:3.5 to 1:4.7, 1:3.5 to 1:4.6, 1:3.5 to 1:4.5, 1:3.5 to 1:4.4, 1:3.5 to 1:4.3, 1:3.5 to 1:4.2, 1:3.5 to 1:4.1, 1:3.5 to 1:4 or less, 1:1 to 1:4, 1:1 to 1:3.5, 1:1 to 1:3, 1:1 to 1:2.5, 1:1 to 1:2, 1:1.5 to 1:4.5, 1:1.5 to 1:4, 1:1.5 to 1:3.5, 1:1.5 to 1:3, 1:1.5 to 1:2.5, 1:2 to 1:4.5, or 1:2.5 to 1:4.5, based on the weight of raw soybean meal, and preferably, it may be higher than 1:3 to lower than 1:5.

In the present invention, when soybean meal is extracted by using an extraction solvent, a solid-liquid separation process using a screw-press or centrifugal extractor may be performed. The screw-press type has a problem of reducing the extraction efficiency, since a separation net is blocked as the operating time passes, and therefore the use of the centrifugal extractor is preferable. When extracting soybean meal by using the centrifugal extractor, the centrifugal force of the centrifuge (rpm) may be for example, 300 g to 1000 g, 400 g to 1000 g, 500 g to 1000 g, 600 g to 1000 g, 700 g to 1000 g, more preferably, 700 to 900 g.

The soybean meal extract solution obtained from the extraction process of the present invention contains a great amount of polysaccharides and a small amount of protein, and therefore it can be used as a culture medium of microorganisms. Since it comprises particularly raffinose and stachyose, etc. abundantly, resources that may be discarded can be recycled, when used for culture or fermentation of lactic acid bacteria.

The soybean meal extract solution may have the sugar concentration of 5 to 20% and/or the protein concentration of 1 to 2% (w/w). Otherwise, the soybean meal extract solution may have the sugar concentration of 6 to 12 Brix and/or the protein concentration of 0.5 to 3% (w/w), or the protein concentration of 0.5 to 2% (w/w).

In the present invention, “residual soybean meal” may have the moisture content of 90% or less, 85% or less, 80 w/w % or less, 75% or less, 70% or less, 65% or less, less than 65%, 64% or less, 63% or less, 62% or less, 61% or less, 60% or less, less than 60%, 59% or less, 58% or less, 55% or less, 50% or less, 45% or less, 40% or less, 35% or less, 30% or less, 25% or less, 20% or less, 15% or less, 10% or less, or 5% or less, and preferably it may be 70% or less, and more preferably it may be 66% or less, and for example, it may be 55 to 66%.

In the present invention, “residual soybean meal” may have sugars or indigestible oligosaccharides of 2% (w/w) or less, 1.9% (w/w) or less, 1.8% (w/w) or less, 1.7% (w/w) or less, less than 1.7% (w/w), 1.6% (w/w) or less, 1.5% (w/w) or less, 1.4% (w/w) or less, 1.3% (w/w) or less, 1.2% (w/w) or less, 1.1% (w/w) or less, 1% (w/w) or less, 0.9% (w/w) or less, 0.8% (w/w) or less, 0.7% (w/w) or less, 0.6% (w/w) or less, 0.5% (w/w) or less, 0.4% (w/w) or less, 0.3% (w/w) or less, 0.2% (w/w) or less, or 0.1% (w/w) or less.

Some sugars or indigestible oligosaccharides are removed by the extraction process of raw soybean meal, and when the concentration of sugars or indigestible oligosaccharides is represented on the basis of 100% by weight of the sugar concentration of raw soybean meal, it may be 99% by weight or less, 98% by weight or less, 97% by weight or less, 96% by weight or less, 95% by weight or less, 90% by weight or less, 85% by weight or less, 80% by weight or less, 75% by weight or less, 70% by weight or less, 65% by weight or less, 60% by weight or less, 55% by weight or less, 50% by weight or less, 45% by weight or less, 40% by weight or less, 35% by weight or less, 30% by weight or less, 25% by weight or less, 20% by weight or less, 15% by weight or less, 10% by weight or less, or 5% by weight or less.

In the present invention, the crude protein content of “residual soybean meal” may be 15% (w/w) or more, 16% (w/w) or more, 17% (w/w) or more, 18% (w/w) or more, 19% (w/w) or more, 20% (w/w) or more, 21% (w/w) or more, or 22% (w/w) or more, on the basis of 65% (w/w) of the moisture content, and may be 55% (w/w) or more, 56% (w/w) or more, 57% (w/w) or more, 58% (w/w) or more, 59% (w/w) or more, 60% (w/w) or more, 61% (w/w) or more, or 62% (w/w) or more, on the basis of the solid content.

The method for preparation of fermented soybean meal of the present invention may comprise a step of fermentation using a microorganism fermenting soybean meal, and specifically, comprises a step of solid culturing by inoculating a soybean meal fermenting microorganism to a fermentation raw material comprising one or more kinds selected from the group consisting of raw soybean meal and residual soybean meal. The method for preparation of fermented soybean meal according to one example of the present invention may further comprise a step of drying and pulverizing the fermented soybean meal obtained in the step of culturing.

In the present invention, a fermentation raw material for preparation of fermented soybean meal may be residual soybean meal obtained by extracting raw soybean meal with an extraction solvent, raw soybean without extraction with an extraction solvent, and a mixture thereof. The raw soybean meal and residual soybean meal as same as described above. In addition, the fermentation raw material may control the crude protein content of fermented soybean meal by appropriately adjusting the mixing ratio of two components, when using the mixture of residual soybean meal and raw soybean meal. For example, when raw soybean meal and residual soybean meal are mixed at 1:1 to 1:1.2, the final crude protein content of fermented soybean meal may be 50 to 54% (w/w). For example, when raw soybean meal and residual soybean meal are mixed at 1:1 to 1:1.4, the final crude protein content of fermented soybean meal may be 50 to 56% (w/w). For example, when raw soybean meal and residual soybean meal are mixed at 1:1.2 to 1:1.4, the final crude protein content of fermented soybean meal may be 53 to 55% (w/w).

In the present invention, “soybean fermenting microorganism” may be a facultative anaerobic bacterium that is a strain having an ability of fermenting soybean meal, and includes for example, Enterococcus sp. strain, Weissella sp. strain, Lactobacillus sp. strain, etc. The facultative anaerobic bacterium may be one or more kinds selected from the group consisting of Enterococcus faecium, Enterococcus faecalis, Weissella koreensis, Pediococcus pentosaceus, Lactobacillus plantarum, Lactobacillus lactis, Lactobacillus reuteri, Lactobacillus brevis, Lactobacillus casei, Lactobacillus fermentum, Lactobacillus bulgaricus, and Lactobacillus acidophilus.

Conventionally, the fermentation of soybean meal is carried out under the aerobic condition which forcibly supply oxygen by using aerobic microorganisms such as Bacillus sp. bacteria, or Aspergillus sp. fungus, or Saccharomyces (beer yeast/baker's yeast) sp. yeast, etc. Since the uniform and sufficient supply of oxygen is difficult in the aerobic process, and the thickness of the fermentation raw material is limited due to a problem of oxygen supply in the aerobic solid fermentation using an aerobic microorganism, and as a result, a fermentation space and equipment are required. In addition, when the fermentation is conducted by using a conventional absolutely anaerobic bacterium such as bifidus, as oxygen transmission should be prevented, rather excessive equipment is required and problems in operation are caused.

In the present invention, as the soybean meal fermenting microorganism, a facultative anaerobic lactic acid bacterium may be used, and in particular, a lactic acid bacterium that grows well in soybean meal extract solution may be used. Accordingly, the biggest problem of the solid fermentation process, the problem of certain supply of oxygen is solved, and block of oxygen is not required, different from the case of using an absolutely anaerobic microorganism, and therefore, large productivity in the fermentation process can be achieved.

Accordingly, the method for preparation of fermented soybean meal according to one example of the present invention comprises a step of performing solid culture of a fermentation raw material using a microorganism fermenting soybean meal, and the solid fermentation may be conducted in an open type solid fermenter in which the movement of oxygen or air is not limited, and preferably, it may not comprise a ventilation process for forcible supply or additional supply of oxygen in the fermentation process. The solid fermentation according to the present invention may be performed by a batch or continuous process, and may use a filling type solid fermenter or a tray type solid fermenter, etc., or a conveyor solid fermenter that the solid fermentation is performed on a conveyor belt may be used, and it is not particularly limited.

For example, an open type continuous solid fermenter may comprise a fermentation chamber which is equipped with an inlet in which a fermentation raw material and/or a lactic acid bacterium is flowed and an outlet out which a fermented product and performs fermentation, a transfer conveyor which is installed inside of the fermentation chamber and transfers the fermentation raw material and/or lactic acid bacterium from the inlet to the outlet, a blade which is equipped to a rotating axis equipped from the inlet to the fermentation chamber in the lateral direction and sets the height of the fermentation raw material in the fermentation chamber, and a scraper which is equipped to a rotating and reverse rotating axis equipped form the outlet to the fermentation chamber in the lateral direction by intervening a bracket and scrapes the fermented product fermented in the fermentation chamber and discharges the fermented product by a predetermined amount.

In addition, in the solid fermentation process of the present invention, when a facultative anaerobic lactic acid bacterium, for example, Enterococcus faecium, Weissella koreeensis, Lactobacillus plantarum, etc. is used, there is a big advantage that lactic acid produced in the fermentation process decreases pH of fermented soybean meal and thereby the contamination of various bacteria can be prevented.

Furthermore, the anaerobic fermentation using lactic acid bacteria generally has a disadvantage of slow rate. To solve the above problem, in the present invention, it is preferable to prepare soybean extract solution comprising sugars and some protein in soybean meal and select a strain showing a high specific growth rate when using it as a culture medium of a microorganism fermenting soybean meal and select extraction and fermentation processes based on this strain.

In case of the solid culture according to one example of the present invention, the strain having an ability of fermenting soybean meal may be inoculated so that the number of bacteria is 10⁵ to 10¹⁰ CFU/g, 10⁵ to 10⁹ CFU/g, 10⁵ to 10⁸ CFU/g, 10⁵ to 10⁷ CFU/g, 10⁵ to 10⁶ CFU/g, 10⁶ to 10¹⁰ CFU/g, 10⁶ to 10⁹ CFU/g, 10⁶ to 10⁸ CFU/g, 10⁶ to 10⁷ CFU/g, 10⁷ to 10¹⁰ CFU/g, 10⁷ to 10⁹ CFU/g, 10⁷ to 10⁸ CFU/g, 10⁸ to 10¹⁰ CFU/g, or 10⁸ to 10⁹ CFU/g right after inoculation to a fermentation raw material, and more preferably it may be inoculated so that the number of bacteria is 10⁶ to 10⁹ CFU/g.

The present solid culture may be performed at a temperature of 25 to 55° C., 25 to 50° C., 25 to 48° C., 25 to 45° C., 25 to 44° C., or 25 to 43° C., for 12 to 48 hours, 12 to 36 hours, 12 to 24 hours, 18 to 48 hours, 18 to 42 hours, 18 to 36 hours, 18 to 30 hours, or 18 to 24 hours. More preferably, the solid culture may be conducted at a temperature range of 25 to 45° C. for 12 to 36 hours. The step of solid culture may conduct culturing under anaerobic conditions, preferably, facultative anaerobic conditions.

The method for preparation of fermented soybean meal according to the present invention, may perform the present culturing by inoculating a microorganism fermenting soybean meal to a fermentation raw material, or inoculating a soybean meal fermenting microorganism to soybean meal extract solution for liquid culturing and adding it to a fermentation raw material. For example, a step of inoculating and culturing a facultative anaerobic soybean meal fermenting microorganism to soybean meal extract solution to obtain a seed culture product and a step of inoculating the seed culture product to a culture raw material to perform the present culturing may be comprised. Specifically, in the present invention, the fermentation can be rapidly progressed, by using lactic acid bacteria which grow well in soybean meal extract solution, by preparing a high concentration of lactic acid bacterial culture solution by using the soybean meal extract solution, and then fermenting the cultured lactic acid bacteria to soybean meal.

According to one example of the present invention, fermented soybean meal may be prepared by adding a seed fermented solution liquid fermented by inoculating a microorganism fermenting soybean meal to soybean meal extract solution to a fermentation raw material. The soybean meal fermenting microorganism may be inoculated to the soybean meal extract solution at a ratio of 0.000001 to 10% by weight, 0.000001 to 5% by weight, 0.000001 to 1% by weight, 0.000001 to 0.1% by weight, 0.000001 to 0.01% by weight, 0.000001 to 0.001% by weight, 0.000001 to 0.0001% by weight, 0.000001 to 0.00001% by weight, 0.00001 to 10% by weight, 0.00001 to 5% by weight, 0.00001 to 1% by weight, 0.00001 to 0.1% by weight, 0.00001 to 0.01% by weight, 0.00001 to 0.001% by weight, 0.00001 to 0.0001% by weight, 0.0001 to 10% by weight, 0.0001 to 5% by weight, 0.0001 to 1% by weight, 0.0001 to 0.1% by weight, 0.0001 to 0.01% by weight, 0.0001 to 0.001% by weight, 0.001 to 10% by weight, 0.001 to 5% by weight, 0.001 to 1% by weight, 0.001 to 0.1% by weight, 0.001 to 0.01% by weight, 0.01 to 10% by weight, 0.01 to 5% by weight, 0.01 to 1% by weight, 0.1 to 10% by weight, 0.1 to 5% by weight, 0.1 to 1% by weight, 0.5 to 10% by weight, 0.5 to 5% by weight, or 0.5 to 1% by weight based on 100% by weight of soybean meal extract solution.

In the liquid culture, the culture temperature may be set according to the optimum growth and development temperature of the soybean meal fermenting microorganism, and for example, it may be cultured at a temperature of 20 to 50° C., 20 to 45° C., 25 to 50° C., 25 to 45° C., 30 to 50° C., 30 to 45° C., 35 to 50° C., 35 to 45° C., 40 to 50° C., or 40 to 45° C.

In the present invention, in order to produce a great deal of soybean meal fermenting microorganism inoculum, a high concentration of culturing method using soybean meal extract solution as a medium has been developed. For this, first, lactic acid bacteria showing high level of growth and development in the soybean meal extract solution prepared by the method. The primary condition is that protein is composed of protein such as trypsin inhibitor (TI) and beta-conglycininin, etc. that are anti-nutritional factors of soybean and is decomposed to a level of small size of protein or peptide so that livestock can smoothly digest soybean meal protein having a relatively high molecular weight, and the secondary condition is a microorganism which can cause fermentation by adapting to soybean meal solid fermentation conditions, for example, moisture content, temperature, pH, etc. General bacteria present in the common natural environment not enterobacteria have many beneficial points for fermentation in particular, as the optimum temperature of growth and development is higher, since there are many bacteria that cannot grow at 40° C. or more. Moreover, as the body temperature is about 41° C. in case of chicken or cow, it may be a characteristic of lactic acid bacteria which is advantageous for survival and proliferation in the actual intestine of livestock.

The Enterococcus faecium SLB130 strain selected according to the investigation method in the present invention showed fast growth and development in soybean meal extract solution, and it was shown that it had a high level of growth and development rate at 40 to 45° C. that was not comparable to Bacillus subtilis or general industrial lactic acid bacteria usually used for soybean meal fermentation. This is an evidence that the selected Enterococcus faecium SLB130 strain effectively decomposes and uses soybean meal organic matters such as oligosaccharides, carbohydrates, proteins, etc. event at 40° C. or more, and in particular, it means that oligosaccharides of soybean which common microorganisms except for lactic acid bacteria cannot use well, for example, sucrose, stachyose, raffinose, etc. may be effectively used as a carbon source. The optimum growth and development temperature of Enterococcus faecium SLB130 strain was 40 to 45° C.

The growth and development of the Enterococcus faecium SLB130 strain in soybean meal extract solution are significantly excellent compared to other lactic acid bacteria or Bacillus subtilis, etc. In addition, the present inventors confirmed that when soybean meal is solid cultured by using the strain, high quality of fermented soybean meal can be prepared in which the digestible absorption rate and feed efficiency are increased, capable of depolymerizing soybean protein by hydrolysis and inactivating trypsin inhibitors, reducing the content of anti-nutritional factors such as indigestible polysaccharides, and controlling the crude protein content according to the mixing ratio of raw soybean meal and extracted residual soybean meal.

In case of lactic acid bacteria, there is a disadvantage in that the usability of soybean oligosaccharides is excellent, while the activity of protease is relatively low, but it is shown that the Enterococcus faecium SLB130 strain has sufficient competitiveness by having an excellent protein decomposition ability. Specifically, the Enterococcus faecium SLB130 strain has an excellent decomposition ability of a polymer peptide, and therefore, as the result of analysis by SDS-PAGE, it could be confirmed that the average molecular weight of protein in fermented soybean meal was decreased, and thus the digestion rate was excellent when used as feed (Example 7).

For example, in the protein distribution of raw soybean meal, based on 100% by weight of the total protein in raw soybean meal, the content having a molecular weight range of less than 25 kD may be 1 to 25% by weight, and the content having a molecular weight range of 25 to less than 50 kD may be 10 to 60% by weight, and the content having a molecular weight range of 50 or more kD may be 30 to 80% by weight.

For example, in the protein distribution of fermented soybean meal in which residual soybean meal is fermented with SLB130 strain, based on 100% by weight of the total protein in fermented soybean meal, the content having a molecular weight range of less than 25 kD may be 60 to 99.9% by weight, 60 to 99% by weight, 60 to 98.5% by weight, or 60 to 98% by weight, and the content having a molecular weight range of 25 to less than 50 kD may be 0.01 to 30% by weight, and the content having a molecular weight range of 50 or more kD may be 0.01 to 10% by weight.

For example, in the protein distribution of fermented soybean meal in which mixed soybean meal, in which residual soybean meal and raw soybean meal are mixed, are fermented with SLB130 strain, based on 100% by weight of the total protein in fermented soybean meal, the content having a molecular weight range of less than 25 kD may be 40 to 80% by weight, and the content having a molecular weight range of 25 to less than 50 kD may be 10 to 45% by weight, and the content having a molecular weight range of 50 or more kD may be 0.01 to 15% by weight.

For example, in the protein distribution of fermented soybean meal in which raw soybean meal is fermented with SLB130 strain, based on 100% by weight of the total protein in fermented soybean meal, the content having a molecular weight range of less than 25 kD may be 30 to 40% by weight, and the content having a molecular weight range of 25 to less than 50 kD may be 47 to 60% by weight, and the content having a molecular weight range of 50 or more kD may be 10 to 20% by weight.

For example, in the protein distribution of fermented soybean meal in which raw soybean meal is fermented with SLB120 strain, based on 100% by weight of the total protein in fermented soybean meal, the content having a molecular weight range of less than 25 kD may be 25 to 40% by weight, and the content having a molecular weight range of 25 to less than 50 kD may be 30 to 50% by weight, and the content having a molecular weight range of 50 or more kD may be 20 to 30% by weight.

The Enterococcus faecium SLB130 strain effectively decomposed and used soybean meal organic matters such as oligosaccharides, carbohydrates, protein, etc., and in particular, used sucrose, stachyose, raffinose, etc. that common microorganisms could not use well as a carbon source. Like this, that microorganisms can be cultured by using an extract in which oligosaccharides that reduce the performance of soybean meal as anti-nutritional factors as a medium can not only reduce cost for preparation of inoculum for soybean meal fermentation, but also make a new value through additional production of industrial lactic acid bacteria powder or lactic acid, amino acid, etc.

It was confirmed that the Enterococcus faecium SLB130 strain has appropriate properties for soybean meal fermentation, by confirming that the optimum growth and development temperature is 40 to 45° C. that is higher than general bacteria (Example 3). The SLB130 strain has beneficial points for fermentation, since the optimum growth and development temperature is high and therefore the contamination of various bacteria can be reduced. In addition, as the body temperature of chicken or cow is about 41° C., it is advantageous for survival and proliferation in the actual intestine of livestock.

The Enterococcus faecium SLB130 strain may have one or more kinds of properties selected from the group consisting of the following (1) to (6):

(1) the optimum growth and development temperature is 40 to 45° C.,

(2) the crude protein content of the soybean fermented product obtained by fermentation with Enterococcus faecium SLB130 strain is higher than 49 to 80% (w/w), 50 to 80% (w/w), higher than 50 to 80% (w/w), higher than 51 to 80% (w/w), 52 to 80% (w/w), 53 to 80% (w/w), higher than 53 to 80% (w/w), 55 to 80% (w/w), 56 to 80% (w/w), higher than 56 to 80% (w/w), 57 to 80% (w/w), 58 to 80% (w/w), 59 to 80% (w/w), or 60 to 80% (w/w), based on 100% by weight of the fermented product,

(3) the trypsin inhibitor content of the soybean meal fermented product obtained by fermentation with Enterococcus faecium SLB130 strain is less than 3.5 mg/g, less than 3 mg/g, 2.5 mg/g or less, less than 2.1 mg/g, 1.5 mg/g or less, less than 1.2 mg/g, 1.1 mg/g or less, 1 mg/g or less, 0.9 mg/g or less, or 0.8 mg/g or less, based on 100% by weight of the fermented product,

(4) the beta-conglycinin of the soybean meal fermented product obtained by fermentation with Enterococcus faecium SLB130 strain is 10,000 ppm or less, 9,000 ppm or less, 8,000 ppm or less, 7,000 ppm or less, 6,000 ppm or less, 5,000 ppm or less, 4,000 ppm or less, 3,000 ppm or less, 2,000 ppm or less, 1,000 ppm or less, 900 ppm or less, 800 ppm or less, 700 ppm or less, 650 ppm or less, less than 620 ppm, 600 ppm or less, 500 ppm or less, 450 ppm or less, less than 420 ppm, 400 ppm or less, 300 ppm or less, 200 ppm or less, or 180 ppm or less, based on 100% by weight of the fermented product,

(5) the indigestible oligosaccharide content of the soybean meal fermented product obtained by fermentation with Enterococcus faecium SLB130 strain is 0.1w/w % or less, 0.09 w/w % or less, 0.08 w/w % or less, 0.07 w/w % or less, 0.06 w/w % or less, 0.05 w/w % or less, 0.04 w/w % or less, or 0.03 w/w % or less, based on 100% by weight of the fermented product,

(6) the lactic acid concentration of the soybean meal fermented product obtained by fermentation with Enterococcus faecium SLB130 strain is 3 w/w % or more, higher than 3.5 w/w %, higher than 3.7 w/w %, 3.8 w/w % or more, higher than 3.9 w/w %, 4 w/w % or more, 4.5 w/w % or more, 4.6 w/w % or more, or 5 w/w % or more, based on 100% by weight of the fermented product,

(7) the pepsin digestion rate of the soybean meal fermented product obtained by fermentation with Enterococcus faecium SLB130 strain is 90w/w % or more, 91w/w % or more, 92w/w % or more, 93w/w % or more, 94w/w % or more, 94.5w/w % or more, 95w/w % or more, 96w/w % or more, or 97w/w % or more,

(8) in the protein distribution of the soybean meal fermented product obtained by fermentation with Enterococcus faecium SLB130 strain, based on 100% by weight of the total protein in fermented soybean meal, the content of protein having a molecular weight range of less than 25 kD is 30 to 99.9% by weight, 30 to 99.5% by weight, 30 to 99% by weight, 30 to 98.5% by weight, 30 to 98% by weight, 30 to 97.5% by weight, or 30 to 97% by weight, and the content of protein having a molecular weight range of 25 to less than 50 kD is 0.01 to 60% by weight, and the content of protein having a molecular weight range of 50 kD or more is 0.01 to 25% by weight.

The Enterococcus faecium SLB130 strain may be cultured continuously or batchwise, and more preferably, continuous culture is preferable. The continuous culture method is a method to put soybean meal extract solution automatically so that certain pH is maintained without separately using a base such as sodium hydroxide or a lactic acid neutralizing agent such as calcium carbonate, based on that the specific growth rate of the Enterococcus faecium SLB130 strain is significantly high.

The batch culture is a method of inoculating the Enterococcus faecium SLB130 strain to soybean meal extract solution to culture it batchwise, and using it as a seed for producing fermented soybean meal when the pH of the culture solution is reduced to 5.5 or less.

The method for preparation of fermented soybean meal according to the present invention may use both liquid culture and solid culture, but it is preferable to include the present culture performing seed culture using soybean meal extract solution and solid fermentation using the seed culture product. To ferment soybean meal in quantity, a large amount of inoculum is needed. In particular, in case of solid fermentation in which the fermentation environment is difficult to control, a large amount of inoculum is required. Thus, preferably, the present invention comprises a two-step culture performing liquid seed culture using soybean meal extract solution and solid culture by inoculating the seed culture product to a fermentation raw material.

In case of liquid culture, since it is not difficult to set the homogeneous environment, sterilization, temperature control, pH control, oxygen supply or oxygen blocking, etc. is easy, and therefore a great amount of inoculum is not required. Commonly, the inoculum size of about 10⁶ to 10⁷ cfu (colony forming unit) per ml or 1-5 v/v % in volume is used. However, to ferment soybean meal using lactic acid bacteria, there are various advantages for using a sufficiently large amount of inoculum. Since the growth and development rate of lactic acid bacteria growing and developing anaerobically (or facultative anaerobically) is slow than aerobic bacteria, or yeast, fungus, etc., a larger amount of inoculum is required to effectively ferment soybean meal.

The seed culture may be performed at 20 to 45° C., 25 to 45° C., 27 to 45° C., 30 to 45° C., 30 to 40° C., 30 to 38° C., 30 to 35° C., 32 to 45° C., 32 to 40° C., 32 to 38° C., 32 to 35° C., 35 to 45° C., or 35 to 40° C., and more preferably it may be performed at 25 to 45° C.

The fermenting microorganism in the seed culture may be inoculated at a ratio of 0.5 to 4.5, 0.5 to 4, 0.5 to 3.5, 0.5 to 3, 0.5 to 2.5, 0.5 to 2, 0.5 to 1.5, 0.5 to 1, 1 to 5, 1 to 4.5, 1 to 4, 1 to 3.5, 1 to 3, 1 to 2.5, 1 to 2, or 1 to 1.5% by weight, based on 100% by weight of the soybean meal extract solution.

The fermented soybean meal according to one example of the present invention may have one or more kinds of properties selected from the following properties:

(1) based on 100% by weight of the fermented product, the lower limit of the crude protein content is 30% (w/w) or more, higher than 30% (w/w), 35% (w/w) or more, 38% (w/w) or more, higher than 38% (w/w), 40% (w/w) or more, 45% (w/w) or more, higher than 46% (w/w), 47% (w/w) or more, 48% (w/w) or more, higher than 48% (w/w), 49% (w/w)or more, higher than 49% (w/w), 50% (w/w) or more, higher than 50% (w/w), 53% (w/w) or more, higher than 53% (w/w), 55% (w/w) or more, 56% (w/w) or more, higher than 56% (w/w), or 60% (w/w) or more, and the upper limit of the crude protein content is 100% (w/w) or less, lower than 100% (w/w), 95% (w/w) or less, 90% (w/w) or less, 85% (w/w) or less, 80% (w/w) or less, 75% (w/w) or less, 70% (w/w) or less, 65% (w/w) or less, or 60% (w/w) or less,

(2) based on 100% by weight of the fermented product, the upper limit of the trypsin inhibitor content is 8 mg/g or less, lower than 8 mg/g, 7 mg/g or less, 6 mg/g or less, 5 mg/g or less, 4.5 mg/g or less, lower than 4.5 mg/g, 4 mg/g or less, 3.5 mg/g or less, lower than 3.5 mg/g, 3 mg/g or less, 2.5 mg/g or less, lower than 2.1 mg/g, 2 mg/g or less, 1.5 mg/g or less, lower than 1.2 mg/g, 1.1 mg/g or less, lower than 1.1 mg/g, 1.0 mg/g or less, lower than 1.0 mg/g, 0.9 mg/g or less, or 0.8 mg/g or less, and the lower limit is 0 mg/g or more, higher than 0 mg/g, 0.0001 mg/g or more, 0.001 mg/g or more, 0.01 mg/g or more, 0.1 mg/g or more, 0.5 mg/g or more, 0.8 mg/g or more, 1 mg/g or more, higher than 1 mg/g, 1.1 mg/g or more, higher than 1.1 mg/g, 1.2 mg/g or more, 1.5 mg/g or more, 2 mg/g or more, 2.1 mg/g or more, higher than 2.1 mg/g, 2.5 mg/g or more, 3 mg/g or more, or 3.5 mg/g or more,

(3) based on 100% by weight of the fermented product, the upper limit of the beta-conglycinin content is 70,000 ppm or less, lower than 70,000 ppm, 68,000 ppm or less, lower than 68,000 ppm, 65,000 ppm or less, lower than 65,000 ppm, 60,000 ppm or less, 50,000 ppm or less, 40,000 ppm or less, 35,000 ppm or less, lower than 35,000 ppm, 30,000 ppm or less, 25,000 ppm or less, 20,000 ppm or less, 15,000 ppm or less, lower than 15,000 ppm, 10,000 ppm or less, 9,000 ppm or less, 8,000 ppm or less, 7,000 ppm or less, 6,000 ppm or less, 5,000 ppm or less, 4,000 ppm or less, 3,000 ppm or less, 2,000 ppm or less, 1,500 ppm or less, lower than 1,500 ppm 1,000 ppm or less, 900 ppm or less, 800 ppm or less, 700 ppm or less, 650 ppm or less, 640 ppm or less, lower than 640 ppm, 630 ppm or less, 620 ppm or less, lower than 620 ppm, 600 ppm or less, 550 ppm or less, 500 ppm or less, 450 ppm or less, 420 ppm or less, 400 ppm or less, 350 ppm or less, 300 ppm or less, 250 ppm or less, 200 ppm or less, or 180 ppm or less, and the lower limit is 0 ppm or more, higher than 0 ppm, 0.0001 ppm or more, 0.001 ppm or more, 0.01 ppm or more, 0.1 ppm or more, 1 ppm or more, 10 ppm or more, 100 ppm or more, 150 ppm or more, 200 ppm or more, 300 ppm or more, or 400 ppm or more,

(4) based on 100% by weight of the fermented product, the upper limit of the indigestible oligosaccharide content is lower than 17.0 mg/g, lower than 7.0 mg/g, 5.0 mg/g or less, 4.0 mg/g or less, 3.0 mg/g or less, lower than 1.70w/w %, 1.5 w/w % or less, lower than 1.1 w/w %, lower than 0.6w/w %, 0.5w/w % or less, 0.4 w/w % or less, 0.3 w/w % or less, 0.2 w/w % or less, 0.15w/w % or less, 0.12 w/w % or less, 0.1 w/w % or less, 0.07 w/w % or less, 0.05 w/w % or less, 0.04 w/w % or less, or 0.03 w/w % or less, and the lower limit is 0 w/w % or more, higher than 0 w/w %, 0.0001 w/w % or more, 0.001 w/w % or more, or 0.01 w/w % or more,

(5) based on 100% by weight of the fermented product, the lower limit of the lactic acid concentration is 0.5% (w/w) or more, 1% (w/w) or more, 1.5% (w/w) or more, 2% (w/w) or more, 2.5% (w/w) or more, 3% (w/w) or more, 3.5% (w/w) or more, 3.8% (w/w) or more, 3.9% (w/w) or more, 4% (w/w) or more, 4.5% (w/w) or more, 4.6% (w/w) or more, 5% (w/w) or more, or 5.1% (w/w) or more, and the upper limit is 10% (w/w) or less, 9.5% (w/w) or less, 9% (w/w) or less, 8.5% (w/w) or less, 8% (w/w) or less, 7.5% (w/w) or less, 7% (w/w) or less, 6.5% (w/w) or less, 6% (w/w) or less, 5.5% (w/w) or less, 5.1% (w/w) or less, 4.6% (w/w) or less, 4% (w/w) or less, 3.9% (w/w) or less, 3.8% (w/w) or less, 3.7% (w/w) or less, or 3.5% (w/w) or less,

(6) the pepsin digestion rate of the soybean meal fermented product is 88w/w % or more, 89 w/w % or more, 90w/w % or more, 91w/w % or more, 92w/w % or more, 93w/w % or more, 94w/w % or more, 94.5w/w % or more, 95w/w % or more, 96w/w % or more, or 97w/w % or more, and

(7) in the protein distribution of the soybean meal fermented product, based on 100% by weight of the total protein in fermented soybean meal, the content of protein having a molecular weight range of less than 251(D is 25 to 99.9% by weight, 25 to 99.5% by weight, 25 to 99% by weight, 25 to 98.5% by weight, 25 to 98% by weight, 25 to 97.5% by weight, or 25 to 97% by weight, and the content of protein having a molecular weight range of 25 to less than 50 kD is 0.01 to 60% by weight, and the content of protein having a molecular weight range of 501(D or more is 0.01 to 30% by weight.

The fermented soybean meal may have the moisture content of 10% (w/w).

Otherwise, the fermented soybean meal according to one example of the present invention may have one or more kinds of properties selected from the following properties:

(1) based on 100% by weight of the fermented product, the crude protein content (w/w %) is 1 time or more, 1.04 times or more, 1.06 times or more, 1.08 times or more, 1.1 times or more, 1.15 times or more, 1.16 times or more, 1.2 times or more, 1.21 times or more, 1.25 times or more, 1.3 times or more, 1.35 times or more, 1.8 times or more, or 2 times or more, compared to the crude protein content (w/w %) before fermentation,

(2) based on 100% by weight of the fermented product, the trypsin inhibitor content (mg/g) is less than 1 time, 0.9 times or less, 0.8 times or less, 0.7 times or less, 0.6 times or less, 0.55 times or less, 0.5 times or less, 0.45 times or less, 0.44 times or less, 0.4 times or less, 0.35 times or less, 0.3 times or less, 0.27 times or less, 0.25 times or less, 0.21 times or less, 0.2 times or less, 0.15 times or less, 0.14 times or less, 0.13 times or less, 0.12 times or less, 0.11 times or less, 0.1 times or less, 0.009 times or less, 0.006 times or less, or 0.003 times or less, compared to the crude protein content (w/w %) before fermentation,

(3) based on 100% by weight of the fermented product, the beta-conglycinin content (ppm) is less than 1 time, 0.9 times or less, 0.8 times or less, 0.7 times or less, 0.6 times or less, 0.5 times or less, 0.4 times or less, 0.3 times or less, 0.21 times or less, 0.2 times or less, 0.1 times or less, 0.25 times or less, 0.22 times or less, 0.01 times or less, 0.009 times or less, 0.008 times or less, 0.007 times or less, 0.006 times or less, 0.005 times or less, 0.004 times or less, 0.003 times or less, 0.002 times or less, or 0.001 times or less, compared to the crude protein content (w/w %) before fermentation,

(4) based on 100% by weight of the fermented product, the indigestible oligosaccharide content (w/w %) is less than 1 time, 0.9 times or less, 0.8 times or less, 0.7 times or less, 0.6 times or less, 0.5 times or less, 0.4 times or less, 0.3 times or less, 0.2 times or less, 0.12 times or less, 0.11 times or less, 0.1 times or less, 0.09 times or less, 0.08 times or less, 0.07 times or less, 0.05 times or less, 0.04 times or less, 0.03 times or less, 0.29 times or less, 0.02 times or less, 0.025 times or less, or 0.01 times or less,

(5) based on 100% by weight of the fermented product, the pepsin digestion rate is 1 time or more, 1.07 times or more, 1.1 times or more, 1.11 times or more, 1,12 times or more, 1.13 times or more, 1.14 times or more, 1.15 times or more, 1.16 times or more, 1.19 times or more, 1.2 times or more, 1.3 times or more, 1.4 times or more, or 1.5 times or more,

(6) based on 100% by weight of the fermented product, the lactic acid concentration (w/w %) is 1 time or more, 1.5 times or more, 2 times or more, 2.5 times or more, or 3 times or more, and

(7) the content of protein less than 25 kD in the fermented product is 1 time or more, 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 3.5 times or more, 3.6 times or more, 3.7 times or more, 3.8 times or more, 3.9 times or more, or 4 times or more, compared to the content of protein less than 25 kD in raw soybean meal before fermentation.

The fermented soybean meal may have the moisture content of 10% (w/w).

It was confirmed that the fermented soybean meal of the present invention comprised a high concentration of lactic acid, and thereby effectively blocked the proliferation of other contaminants (Table 8). The soybean meal fermenting microorganism secrets lactic acid in the fermentation process, and this shows an effect of decreasing pH of a medium and consequently plays a role of blocking the proliferation of other contaminants.

According to another example of the present invention, a method for preparation of fermented soybean meal, which comprises a step of extracting raw soybean meal to obtain soybean meal extract solution and residual soybean meal; and a fermentation step of inoculating and culturing a microorganism fermenting soybean meal or culture product of the soybean meal fermenting microorganism to a fermentation raw material comprising residual soybean meal or a mixture of residual soybean meal and raw soybean meal, and controls the crude protein content of fermented soybean meal by adjusting the content of residual soybean meal comprised in the fermentation raw material in the fermentation step, may be provided.

The fermentation raw material may be one or more kinds selected form the group consisting of (1) raw soybean meal, (2) residual soybean meal after extraction, and (3) a mixture of (1) and (2). The residual soybean meal obtained after extraction has the high crude protein content as protein is concentrated, and therefore, in case of the mixed fermentation raw material of (3), as the content of residual soybean meal becomes higher, the crude protein content of fermented soybean meal is increased, and thus, the final crude protein content of fermented soybean meal may be appropriately controlled by adjusting the mixing ratio of raw soybean meal and residual soybean meal.

For example, to solid culture only the (2) residual soybean meal after extraction as a fermentation raw material is suitable for production of a high concentration of crude protein product, and it may be used as an alternative for fishmeal used for feed for hatchery fish such as shrimp or eel, flatfish, abalone. For example, to solid culture only the (1) raw soybean meal as a fermentation raw material is suitable for use for adult livestock such as mother pig and growing pig, broiler chicken and layer chicken, etc.

For example, to solid culture the mixed (1) raw soybean meal and (2) residual soybean meal after extraction is suitable for young animals such as piglet, etc., since the crude protein content is high.

In the mixture in which the (1) raw soybean meal and (2) residual soybean meal after extraction are mixed, the raw soybean meal and residual soybean meal after extraction may be mixed at a weight ratio of 1:10 to 10:1, 1:9 to 9:1, 1:8 to 8:1, 1:7 to 7:1, 1:6 to 6:1, 1:5 to 5:1, 1:4 to 4:1, 1:3 to 3:1, 1:2 to 2:1, 1:1.8 to 1.8:1, 1:1.6 to 1.6:1, 1:1.5 to 1.5:1, 1:1 to 1:10, 1:1 to 1:9, 1:1 to 1:8, 1:1 to 1:7, 1:1 to 1:6, 1:1 to 1:5, 1:1 to 1:4, 1:1 to 1:3. 1:1 to 1:2, or 1:1 to 1:5. More preferably, they may be mixed at a weight ratio of 1:1 to 1:1.5.

Other one example of the present invention is to provide a feed composition comprising fermented soybean meal. The feed composition may be used as feed for one or more kinds selected from the group consisting of pig, cow, chicken, duck, goat, sheep, dog and cat. Otherwise, the feed composition may be used as the feed composition for adult livestock, piglet or hatchery fish, according to the crude protein content.

In the feed composition for adult livestock, it is preferable that the crude protein content is about 48 to 50%, and it may be prepared by mass production in a short time, by using only the raw soybean meal as a fermentation raw material at a high drying temperature at which it is about 63-65% when measured by KOH solubility. The feed composition for adult livestock can raise economic feasibility by using only raw soybean meal as a fermentation raw material.

For the feed composition for young animals, it is preferable to improve the quality of fermented soybean meal such as increasing the crude protein content and removing anti-nutritional factors and increasing the digestive absorption rate of amino acids, etc., so as to be appropriate for rapid growth of young livestock. The feed composition for young animals may have properties of removing anti-nutritional factors and developing epithelial cells and increasing intestinal beneficial bacteria, in order to facilitate the growth of young unit animal such as piglet, young chick, early broiler chicken, etc. For this, raw soybean meal and residual soybean meal are mixed and used together as a fermentation raw material, and it may be prepared by drying at a low temperature so that it is about 65-70% when measured by KOH solubility, and the crude protein content is 52 to 54%, preferably 53% or more and the crude protein content is higher than the feed for adult livestock.

The feed composition for hatchery fish is to alternate fishmeal used for hatchery fish feed, and for using fermented soybean meal as feed for hatchery fish, the crude protein content is important, but the removal of indigestible oligosaccharides such as raffinose or stachyose is the most important. The reason is because it is preferable to remove indigestible components in advance as much as possible, as fish has short intestine and therefore the digestion time is not enough. For this, only residual soybean meal is used as a fermentation raw material, and the drying temperature is low as much as possible so that it is 70% or more measured by KOH solubility for thermal denaturation, and it may be prepared so that the crude protein content is 60% or more, preferably 60 to 65%.

The hatchery fish may be one or more kinds selected from the group consisting of for example, mackerel, flatfish, halibut, rockfish, trout, salmon, catfish, eel, tilapia, seabass, Nile catfish, white leg shrimp, giant tiger prawn, fleshy prawn, Kuruma shrimp and abalone, but not limited thereto.

The present invention suggests a technical basis which can prepare various fermented soybean meal to suit applications by extracting aqueous sugars including indigestible oligosaccharides using a small amount of extract solution in soybean meal first and thereby mixing residual soybean meal remained after extraction and raw soybean meal alone or in an appropriate ratio.

It was shown that in the fermented soybean according to the present invention, the digestive absorption rate was increased, as anti-nutritional factors were sufficiently inactivated and soybean protein was considerably decomposed even within the short fermentation time. In addition, a method for culturing the lactic acid bacterium in quantity by using the soybean meal extract solution obtained as a byproduct of the process was devised, thereby creating new added values.

### Advantageous Effects

The present invention provides a method for producing fermented soybean meal having high cost competitiveness in the animal feed market as an anaerobic fermentation process technology which can produce fermented soybean meal with relatively simple process equipment and low cost, as an alternative to the conventional aerobic fermentation process which requires a lot of facility and operating costs.

The fermentation process in the present invention can be stably operated, since the fermentation time is considerably short and the possibility of contamination is low, compared to the conventional lactic acid bacteria fermentation process. The fermented soybean meal prepared by the process of the present invention has enough competitiveness compared to that produced in the conventional aerobic fermentation process in quality. In particular, the indigestible oligosaccharides which disturb digestive absorption of livestock and thereby significantly reduce the feed efficiency of soybean meal can be excellently removed in the process of the present invention. In addition, the fermented soybean meal segmented according to livestock species and objects by using an extraction process can be produced.

In the present invention, the soybean extract solution which can be discarded nearly is recycled as lactic acid bacteria culture medium, and thereby the cost can be reduced, and also environment-friendly means without process waste are provided. Moreover, since products such as industrial lactic acid bacteria powder or lactic acid, etc. by using extra extract solution, added values can be created. In addition, a technological basis for producing various fermented products having an industrial value through fermentation of soybean extract solution by using various microorganisms is proposed.

In addition, the technology of investigating bacteria using aqueous soybean meal extract solution as a microbial medium like the present invention provides innovative technological foundation which can effectively recycle various organic byproducts which can increase the industrial value during fermentation in addition to soybean meal by a low cost of anaerobic process.

The fermented soybean meal according to the present invention showed that anti-nutritional factors were inactivated enough and soybean protein was significantly degraded during the short fermentation time and therefore the digestive absorption became high. In addition, a method for culturing the lactic acid bacteria in quantity using the soybean extract solution obtained by process byproducts is devised, thereby creating new added vales.

The purpose of the present invention is to produce lactic acid bacteria fermented soybean meal using a lactic acid bacterium. The lactic acid bacteria are economical compared to the aerobic fermentation process using Bacillus subtilis or a fungus, but there is a problem in the process such as anaerobic condition, etc. To overcome this, in the present invention, a high concentration of lactic acid bacteria culture solution is prepared from lactic acid bacteria which grow well in soybean meal extract solution by using the soybean extract solution, and then the cultured lactic acid bacteria are inoculated to soybean meal and fermented, so that the fermentation is rapidly progressed.

The present invention is to increase the digestion rate of defatted soybean meal that is a major raw material of livestock feed and to reduce anti-nutritional factors, thereby increasing the availability of nutrients.

The fermentation process in the present invention can be stably operated, since the fermentation time is considerably short and the possibility of contamination is low, compared to the conventional lactic acid bacteria fermentation process. In addition, the present invention can produce fermented soybean meal, which indigestible oligosaccharides which disturb digestive absorption of livestock and thereby significantly reduce the feed efficiency of soybean meal can be easily removed in and is segmented according to livestock species and objects by using an extraction process.

Thus, the digestion rate of the defatted soybean meal that is a major raw material of livestock feed can be increased and the anti-nutritional factors can be reduced, thereby increasing the availability of nutrients.

### BRIEF DESCRIPTION OF DRAWINGS

FIG. 1 is the result of analyzing the protein in the soybean meal before fermentation and soybean meal after fermentation by SDS-PAGE, and A of FIG. 1 is the result of SDS-PAGE analysis of the protein sample of the raw soybean meal before fermentation, and B of FIG. 1 is the result of SDS-PAGE analysis of the protein sample of the fermented soybean meal in which the raw soybean meal is fermented by using SLB120 strain (Example 4-2), and C to E of FIG. 1 are the results of SDS-PAGE of the protein sample of the fermented soybean meal in which the residual soybean meal is fermented by using SLB130 strain (Example 5-1), and F of FIG. 1 is a size marker and each line represents the sizes of 250, 150, 100, 75, 50, 37, 25, 20, 15, and 10 kD in order from the top, and G of FIG. 1 is the result of SDS-PAGE analysis of the protein sample of the fermented soybean meal in which the raw soybean meal is fermented by using SLB130 strain (Example 4-1).

### MODE FOR INVENTION

Hereinafter, the present invention will be described in more detail by the following examples. However, these examples are intended to illustrate the present invention only, but the scope of the present invention is not limited by these examples.

### EXAMPLE 1

### Optimization of Soybean Meal Extraction Conditions

### 1-1: Weight Ratio of Raw Soybean Meal and Extraction Solvent

Water at a room temperature was used as an extraction solvent of soybean meal. Soybean meal extraction solution was prepared by varying the weight ratio of raw soybean meal and the extraction solvent to 2.5, 3, 4, 5, 7, 10 and 20 of the extraction solvent weight based on 1 of the soybean meal weight.

Specifically, water of pH 7.0 and temperature 40° C. was added to soybean meal 100 g (moisture 12%, crude protein content 46%) and it was extracted for 30 min and filtered with a 50 mesh sieve to obtain filtrate which was used as soybean meal extract solution.

The sugar concentration (Brix) of the soybean meal extract solution was measured by using a saccharometer. In addition, the protein content of the extract solution obtained after extraction was quantified by Bradford method. The crude protein content of residual protein was calculated by excluding the crude protein content extracted by extraction (protein content in the extract solution) from the crude protein content present in the raw soybean meal.

The result of analysis of the sugar content of the soybean meal extract solution and the protein content of the residual soybean meal was shown in the following Table 1. In Table 1, 1 Bris was defined as the g number of sugars comprised in 100 g of solution and the amount of protein was expressed as mass (g) of protein contained per 100 g of extract solution.

TABLE 1

| | Sugar con- | | | |
|---|---|---|---|---|
| Mass ratio | centration | Total sugars | Total protein | Protein of |
| of soybean | of extract | in extract | in extract | residual |
| meal:extrac- | solution | solution | solution | soybean meal |
| tion solvent | (Brix) | (g) | (g) | (w/w %) |
| 1:2.5 | 12.0 | 15.6 | 1.2 | 53.8 |
| 1:3 | 10.0 | 18.0 | 1.3 | 55.4 |
| 1:4 | 7.0 | 19.6 | 1.4 | 56.5 |
| 1:5 | 4.5 | 17.6 | 1.4 | 55.1 |
| 1:7 | 3.0 | 17.9 | 1.5 | 55.2 |
| 1:10 | 2.2 | 19.14 | 1.6 | 56.0 |
| 1:20 | 1.2 | 22.46 | 1.7 | 58.4 |

As a result, it was confirmed that when the soybean meal and extraction solvent were extracted at a weight ratio of 1:4, the optimum conditions of the soybean meal extract solution were achieved, which were 7 Brix of the sugar concentration of the soybean meal extract solution, 19.6 g of the total sugars of the entire extract solution, and 1.4% (w/w) of the total protein concentration in the extract solution. When a larger amount of solvent was used, the total amount of indigestible sugars extracted from the raw soybean meal was increased, but the amount of aqueous protein eluted together was also increased, and this reduced the crude protein content of the final fermented soybean meal and also reduced the output of fermented soybean meal. In addition, in consideration of a disadvantages in the process, when a large amount of extraction solvent was used, it could be seen that the weight ratio of about 1:4 was preferable.

1-2: Establishment of pH Condition of Extraction Solvent

To establish the optimum extraction condition for preparation of the soybean meal extract, after setting pH and temperature conditions of the extraction solvent variously, components of the soybean meal extract solution obtained by extracting soybean meal were analyzed. The result was shown in Table 2. The extraction time was 30 min, and in case that the temperature of the extraction solvent was 60° C. or 80° C., when the extraction time was 30 min, the extraction was not good, and thus it was extracted for 15 min.

TABLE 2

| | | | | Sugar | Sugar | | Protein |
|---|---|---|---|---|---|---|---|
| | Extraction | | Soybean | concentration | content of | Protein | of residual |
| | solvent | Extraction | meal:extraction | of extract | extraction | of extract | soybean |
| | temperature | time | solvent ratio | solution | solution | solution | meal |
| pH | (° C.) | (min) | (w/w) | (Brix) | (g) | (g) | (w/w %) |
| 3 | 40 | 30 | 1:4 | 6.7 | 18.8 | 1.2 | 56.0 |
| 4 | 40 | 30 | 1:4 | 6.9 | 19.3 | 1.3 | 56.3 |
| 5 | 40 | 30 | 1:4 | 6.8 | 19.0 | 1.4 | 56.1 |
| 7 | 40 | 30 | 1:4 | 6.9 | 19.3 | 1.5 | 56.2 |
| 9 | 40 | 30 | 1:4 | 6.7 | 18.8 | 2.2 | 55.4 |
| 10 | 40 | 30 | 1:4 | 6.8 | 19.0 | 3.8 | 54.7 |
| 7.0 | 40 | 30 | 1:4 | 6.9 | 19.3 | 1.5 | 56.2 |
| 7.0 | 60 | 15 | 1:4 | 6.9 | 19.3 | 1.5 | 56.2 |
| 7.0 | 80 | 15 | 1:4 | 7.1 | 19.9 | 1.8 | 56.4 |

As the result of analysis of components, it could be seen that it was preferable that the pH of the extraction solvent was 8 or less, since problems that the crude protein content of the final fermented soybean meal was lowered and the output was reduced finally, as the elution of protein of soybean meal was increased as the pH of the extraction solvent was increased and thereby the crude protein content of the residual soybean meal was lowered.

### EXAMPLE 2

### Crude Protein Content of Soybean Meal Extract Solution According to pH and Temperature Conditions of Extraction Solvent

By measuring the crude protein content of the soybean meal extract solution according to pH and temperature conditions of the extraction solvent, the optimum conditions of the extraction solvent for controlling the crude protein content of the residual soybean meal were established.

Specifically, water was used as the extraction solvent, and hydrochloric acid was added as a pH regulator for adjusting the pH of the extraction solvent. The weight ratio of the raw soybean meal and extraction solvent was 1:4, and after extracting raw soybean meal using extraction solvents of the pH and temperature conditions of Table 3, the crude protein content of the soybean meal extract solution was measured.

TABLE 3

| | | | | Sugar | Sugar | | Protein |
|---|---|---|---|---|---|---|---|
| | Soybean | | | concentration | content of | Protein | of residual |
| | meal:extraction | | | of extract | extract | of extract | soybean |
| | solvent ratio | | Temperature | solution | solution | solution | meal |
| Classification | (w/w) | pH | (° C.) | (Brix) | (g) | (g) | (w/w %) |
| Example2-1 | 1:4 | 3.5 | 27 | 6.8 | 19.0 | 1.4 | 56.1 |
| Example2-2 | 1:4 | 7.8 | 27 | 6.8 | 19.0 | 1.5 | 56.0 |
| Example2-3 | 1:4 | 7.8 | 60 | 6.9 | 19.3 | 1.6 | 56.1 |
| Example2-4 | 1:4 | 7.8 | 80 | 6.9 | 19.3 | 1.9 | 56.0 |

As a result, it could be seen that it was preferable that the pH of the extraction solvent was 4 or less, as the crude protein content was increased, when pH was high.

In addition, it could be seen that it was preferable as the temperature of the extraction solvent was low, since the extracted crude protein content was increased as the temperature of the extraction solvent was increased. In particular, it was confirmed that even when the temperature of the extraction solvent was set to 25 to less than 40° C. in case that pH was 4 or less, anti-nutritional factors such as indigestible oligosaccharides, etc. were removed sufficiently at the equal level to the high temperature extraction. In other words, it was confirmed that the efficient removal of anti-nutritional factors of raw soybean meal was possible, since in case that pH was 4 or less, even when the temperature of the extraction solvent was relatively low, there was few effects on the sugar content extracted from the raw soybean meal and the protein content extracted from the raw soybean meal was small.

### EXAMPLE 3

### Separation and Identification of Soybean Meal Fermenting Microorganism

### 3-1: Preparation of Soybean Meal Extract Solution

To establish a soybean meal fermenting microorganism of which growth and development were excellent in the soybean meal extract solution obtained by extracting raw soybean meal with an extraction solvent, soybean meal extract solution was prepared and used as a medium.

Specifically, under the optimum extraction conditions established in Example 1, the soybean meal extract solution was prepared as follows. Water of 25° C. of which pH was adjusted to 3.5 by adding hydrochloric acid was used as the extraction solvent.

The extraction solvent was added to the soybean meal and then it was stirred enough, and in 10 min, it was filtered with a 50 mesh sieve to obtain the soybean meal extract solution.

3-2: Separation of Soybean Meal Fermenting Microorganism using Soybean Meal Extract Solution

To separate a microorganism having the ability of fermenting soybean meal, the followings were carried out.

A candidate strain having the ability of fermenting soybean meal was extracted by using an MRS lactic acid bacteria selective medium, from the soybean meal which water was added in and was left outside for a week or more.

That the soybean meal extract solution prepared in Example 3-1 was filtered by using a sterilized membrane filter of pore size 0.22 um was used as a medium, and the candidate strain was liquid cultured overnight in the MRS medium, and as a control group, a Bacillus strain was added to each medium at the concentration of 1%, which was liquid cultured overnight in Nutrient broth medium. Each microorganism was grown and developed at the temperature of 40° C. and 45° C. and cultured for 12 hours, and then the viable cell count was measured.

3-3: Identification of Soybean Meal Fermenting Microorganism

According to the result of measurement of the viable cell count, the microorganism showing the high growth and development speed was selected, and 16s rRNA of the selected microorganism was amplified with the primer pair having SEQ ID NOs: 2 and 3 of the following Table 4, and through sequencing, the microorganism identification was conducted. The result was same as 16s rRNA, the sequence of Table 4 below, and the identification of the strain was finally completed by using this sequence information and multiply comparing with BLAST program.

TABLE 4

| Name | Sequence listing (5′→3′) |
|---|---|
| Entero- | ACGCGGGCGGCGTGCCTAATACATGCAAGTCGTACGCT |
| coccus | TCTTTTTCCACCGGAGCTTGCTCCACCGGAAAAAGAGG |
| faecium | AGTGGCGAACGGGTGAGTAACACGTGGGTAACCTGCCC |
| SLB130 | ATCAGAAGGGGATAACACTTGGAAACAGGTGCTAATAC |
| (SEQ ID | CGTATAACAATCGAAACCGCATGGTTTTGATTTGAAAG |
| NO: 1) | GCGCTTTCGGGTGTCGCTGATGGATGGACCCGCGGTGC |
| | ATTAGCTAGTTGGTGAGGTAACGGCTCACCAAGGCCAC |
| | GATGCATAGCCGACCTGAGAGGGTGATCGGCCACATTG |
| | GGACTGAGACACGGCCCAAACTCCTACGGGAGGCAGC |
| | AGTAGGGAATCTTCGGCAATGGACGAAAGTCTGACCGA |
| | GCAACGCCGCGTGAGTGAAGAAGGTTTTCGGATCGTAA |
| | AACTCTGTTGTTAGAGAAGAACAAGGATGAGAGTAACT |
| | GTTCATCCCTTGACGGTATCTAACCAGAAAGCCACGGC |
| | TAACTACGTGCCAGCAGCCGCGGTAATACGTAGGTGGC |
| | AAGCGTTGTCCGGATTTATTGGGCGTAAAGCGAGCGCA |
| | GGCGGTTTCTTAAGTCTGATGTGAAAGCCCCCGGCTCA |
| | ACCGGGGAGGGTCATTGGAAACTGGGAGACTTGAGTGC |
| | AGAAGAGGAGAGTGGAATTCCATGTGTAGCGGTGAAAT |
| | GCGTAGATATATGGAGGAACACCAGTGGCGAAGGCGG |
| | CTCTCTGGTCTGTAACTGACGCTGAGGCTCGAAAGCGT |
| | GGGGAGCAAACAGGATTAGATACCCTGGTAGTCCACGC |
| | CGTAAACGATGAGTGCTAAGTGTTGGAGGGTTTCCGCC |
| | CTTCAGTGCTGCAGCTAACGCATTAAGCACTCCGCCTG |
| | GGGAGTACGACCGCAAGGTTGAAACTCAAAGGAATTG |
| | ACGGGGGCCCGCACAAGCGGTGGAGCATGTGGTTTAAT |
| | TCGAAGCAACGCGAAGAACCTTACCAGGTCTTGACATC |
| | CTTTGACCACTCTAGAGATAGAGCTTCCCCTTCGGGGG |
| | CAAAGTGACAGGTGGTGCATGGTTGTCGTCAGCTCGTG |
| | TCGTGAGATGTTGGGTTAAGTCCCGCAACGAGCGCAAC |
| | CCTTATTGTTAGTTGCCATCATTCAGTTGGGCACTCTA |
| | GCAAGACTGCCGGTGACAAACCGGAGGAAGGTGGGGAT |
| | GACGTCAAATCATCATGCCCCTTATGACCTGGGCTACA |
| | CACGTGCTACAATGGGAAGTACAACGAGTTGCGAAGTC |
| | GCGAGGCTAAGCTAATCTCTTAAAGCTTCTCTCAGTTC |
| | GGATTGCAGGCTGCAACTCGCCTGCATGAAGCCGGAAT |
| | CGCTAGTAATCGCGGATCAGCACGCCGCGGTGAATACG |
| | TTCCCGGGCCTTGTACACACCGCCCGTCACACCACGAG |
| | AGTTTGTAACACCCGAAGTCGGTGAGGTAACCTTTTTG |
| | GAGCCAGCCGCCTAAGGTGGGATAGATGATGGGGGTGA |
| | AGTCGTAACAAGGTAGCCGTATCGGAAGGTGCGGCTGG |
| | ATCCCC |
| universal | GGTTAGATACCCTGGTA |
| forward | |
| primer 785F | |
| (SEQ ID | |
| NO: 2) | |
| universal | CCGTCAATTCMTTTRAGTTT |
| reverse | |
| primer 785F | |
| (SEQ ID | |
| NO: 3) | |

The selected Enterococcus faecium was named Enterococcus faecium SLB130, and was deposited to the international depository institution under Budapest Treaty, Korean Collection for Type Culture on June 29, 2018 and the accession number, KCTC13566BP was given.

3-4: Condition of Growth and Development of Soybean Meal Fermenting Microorganism

The result of growth and development of Enterococcus faecium SLB130 strain in the soybean meal extract solution was shown in Table 5 and Table 6, and as a control group, Enterococcus faecium SLB120 (accession number: KCTC12868BP) and Bacillus megaterium were used. The OD value and viable cell count of the culture solution and the pH of the culture solution measured after culturing the strain for 12 hours were shown in Table 5 and Table 6. Table 5 showed the result of growth and development in the 40° C. soybean meal extract solution, and Table 6 showed the result of growth and development in the 45° C. soybean meal extract solution.

TABLE 5

| | | | Viable cell count |
|---|---|---|---|
| Strain name | OD600 | pH | (cfu/ml) |
| Enterococcus faecium SLB130 | 3.42 | 4.8 | 6.3 × 10⁹ |
| Enterococcus faecium SLB120 | 2.65 | 5.5 | 1.4 × 10⁸ |
| Bacillus megaterium | 0.32 | 6.5 | 2.1 × 10⁶ |

TABLE 6

| | | | Viable cell count |
|---|---|---|---|
| Strain name | OD600 | pH | (cfu/ml) |
| Enterococcus faecium SLB130 | 3.31 | 4.9 | 6.2 × 10⁹ |
| Enterococcus faecium SLB120 | 1.73 | 6.1 | 4.2 × 10⁷ |
| Bacillus megaterium | 0.19 | 6.5 | 1.2 × 10⁶ |

As a result, as shown in Table 5 and Table 6, the selected strain showed excellent growth and development compared to the control group at the temperature of 40° C. and 45° C., compared to the conventionally separated Enterococcus faecium SLB120 which was used as the control group and bacillus subtilis Bacillus megaterium known to secret a great deal of organic matter decomposing enzymes. The optimum temperature of growth and development of the selected strain SLB130 was shown as 40 to 45° C.

### EXAMPLE 4

### Preparation of Fermented Soybean Meal using Raw Soybean Meal

### Example 4-1 Preparation of Fermented Soybean Meal using Raw Soybean Meal (1)

Using the soybean meal fermenting microorganism selected in Example 3, the result of fermentation under various soybean meal conditions was measured.

Specifically, using the raw soybean meal before extraction used in Example 1 as a fermentation raw material, the soybean meal fermenting microorganism selected in Example 3, Enterococcus faecium SLB130 (KCTC13566BP) was inoculated to prepare fermented soybean meal. Specifically, the fermentation was conducted by inoculating Enterococcus faecium SLB120 at the concentration of 2.1×10⁸ to the fermentation raw material in an open type tray solid fermenter and starting the fermentation at the temperature of 30° C., thereby increasing the temperature by 40° C., and carrying out the fermentation for 24 hours. During the fermentation process, oxygen was not supplied. Then, it was dried in a tray drier of 50° C. temperature, and the components and characteristic of the fermented soybean meal were analyzed and shown in Table 8. In addition, the components and characteristics of the raw soybean meal were analyzed and shown in Table 7.

In Table 7 and Table 8, the lactic acid concentration is represented by the amount of lactic acid comprised in the fermentation raw material or fermented product as w/w %. The KOH solubility means w/w % of quantifying the amount of the crude protein after extracting the fermentation raw material or fermented product with 0.2% KOH and dividing it by the total crude protein content. This is used as an index for the content of protein which can be absorbed in small intestines. The pepsin digestion rate is the value calculated by the following equation, after adding the fermentation raw material or fermented product to 0.2% pepsin hydrochloric acid solution and digesting for 16 hours in a 45° C. constant-temperature water bath and counting the crude protein content of undigested products.

Pepsin digestion rate (%)=(A−B)/A×100 [Equation 1]

(A: Crude protein content (g),

B: Crude protein content in undigested products (g))

Example 4-2: Preparation of Fermented Soybean Meal using Raw Soybean Meal (2)

With the same method except for using Enterococcus faecium SLB120 (KCTC12868BP), instead of Enterococcus faecium SLB130 (KCTC13566BP) used in Example 4-1, fermented soybean meal was prepared. Then, it was dried in a tray drier of 50° C. temperature, and the components and characteristics of fermented soybean meal were analyzed and shown in Table 8. In addition, the components and characteristics of raw soybean meal used as a fermentation raw material were analyzed and shown in Table 7.

### EXAMPLE 5

### Preparation of Fermented Soybean Meal using Residual Soybean Meal

### Example 5-1: Preparation of Fermented Soybean Meal using Residual Soybean Meal (1)

By using Enterococcus faecium SLB130 (KCTC13566BP), with the substantially same method with Example 4, fermented soybean meal was prepared, but the residual soybean meal extracted with an extraction solvent was used instead of the raw soybean meal of Example 4 as a fermentation raw material to prepare fermented soybean meal. Then, it was dried in a tray drier of 50° C. temperature, and the components and characteristics of fermented soybean meal were analyzed and shown in Table 8. In addition, the components and characteristics of residual soybean meal used as a fermentation raw material were analyzed and shown in Table 7.

Example 5-2: Preparation of Fermented Soybean Meal using Residual Soybean Meal (2)

With the same method except for using Enterococcus faecium SLB120 (KCTC12868BP), instead of Enterococcus faecium SLB130 (KCTC13566BP) used in Example 5-1, fermented soybean meal was prepared by using the residual soybean meal obtained in Example 2 as a fermentation raw material. Then, it was dried in a tray drier of 50° C. temperature, and the components and characteristics of fermented soybean meal were analyzed and shown in Table 8.

### EXAMPLE 6

### Preparation of Fermented Soybean Meal using Mixed Soybean Meal

### Example 6-1: Preparation of Fermented Soybean Meal using Mixed Soybean Meal (1)

By using Enterococcus faecium SLB130 (KCTC13566BP), with the substantially same method with Example 4, fermented soybean meal was prepared, but the mixed soybean meal raw material in which the raw soybean meal of Example 1 and the residual soybean meal of Example 2 were mixed at a weight ratio of 0.7:1 was used instead of the raw soybean meal of Example 4 as a fermentation raw material to prepare fermented soybean meal. Then, it was dried in a tray drier of 50° C. temperature, and the components and characteristics of fermented soybean meal were analyzed and shown in Table 8. In addition, the components and characteristics of the mixed soybean meal raw material used as a fermentation raw material were analyzed and shown in Table 7.

### Example 6-2: Preparation of Fermented Soybean Meal using Mixed Soybean Meal (2)

With the same method except for using Enterococcus faecium SLB120 (KCTC12868BP), instead of Enterococcus faecium SLB130 (KCTC13566BP) used in Example 6, fermented soybean meal was prepared by using the mixed soybean meal raw material in which the raw soybean meal and the residual soybean meal were mixed at a weight ratio of 0.7:1 a fermentation raw material. Then, it was dried in a tray drier of 50° C. temperature, and the components and characteristics of fermented soybean meal were analyzed and shown in Table 8. In addition, the components and characteristics of the mixed raw material used as a fermentation raw material were analyzed and shown in Table 7.

TABLE 7

| | Item | | |
|---|---|---|---|
| | Raw | Residual | |
| | soybean | soybean | Mixed raw |
| | meal | meal | material |
| Moisture content (w/w %) | 12 | 65 | 40 |
| pH | 6.5 | 6.7 | 6.6 |
| Crude protein content (w/w %) | 46 | 30 | 38 |
| Trypsin inhibitor (mg/g) | 8.0 | 2.1 | 4.5 |
| Beta-conglycininin (ppm) | 70,000 | 65,000 | 68,000 |
| Lactic acid concentration (w/w %) | 0 | 0 | 0 |
| Indigestible oligosaccharide | 1.70 | 0.6 | 1.1 |
| content (w/w %) | | | |
| KOH solubility (w/w %) | 80 | 82 | 81 |
| Pepsin digestion rate (w/w %) | 82.7 | 85 | 83 |
| Lactic acid bacteria viable cell | ND | ND | ND |
| (cfu/g) | | | |
| General bacteria (cfu/g) | 5.4 × 10⁵ | 0.5 × 10⁵ | 4.8 × 10⁵ |

TABLE 8

| | Example | Example | Example | Example | Example | Example |
|---|---|---|---|---|---|---|
| Item | 4-2 | 5-2 | 6-2 | 4-1 | 5-1 | 6-1 |
| Moisture content | 10 | 10 | 10 | 10 | 10 | 10 |
| (w/w %) | | | | | | |
| pH | 6.0 | 5.2 | 5.7 | 5.8 | 5.1 | 5.4 |
| Crude protein content | 49 | 56 | 51 | 50 | 60 | 53 |
| (w/w %) | | | | | | |
| Trypsin inhibitor | 3.5 | 1.2 | 2.1 | 1.1 | 0.8 | 1.0 |
| (mg/g) | | | | | | |
| Beta-conglycininin | 35,000 | 640 | 1,500 | 620 | 180 | 420 |
| (ppm) | | | | | | |
| Lactic acid | 3.4 | 3.9 | 3.7 | 3.8 | 5.1 | 4.6 |
| concentration (w/w %) | | | | | | |
| Indigestible | 0.15 | 0.07 | 0.12 | 0.05 | 0.03 | 0.04 |
| oligosaccharide | | | | | | |
| content (w/w %) | | | | | | |
| KOH solubility | 70.1 | 70.7 | 70.4 | 73.0 | 77.4 | 76.4 |
| (w/w %) | | | | | | |
| Pepsin digestion rate | 88.3 | 94.7 | 92.1 | 94.2 | 97.7 | 95.5 |
| (w/w %) | | | | | | |
| Lactic acid bacteria | 1.1 × 10\hat{ }8 | 1.4 × 10\hat{ }8 | 1.2 × 10\hat{ }8 | 2.1 × 10\hat{ }8 | 3.8 × 10\hat{ }9 | 2.3 × 10\hat{ }8 |
| viable cell (cfu/g) | | | | | | |
| General bacteria | 5.8 × 10\hat{ }4 | 5.4 × 10\hat{ }4 | 5.6 × 10\hat{ }4 | 3.4 × 10\hat{ }4 | 3.1 × 10\hat{ }3 | 1.2 × 10\hat{ }4 |
| (cfu/g) | | | | | | |

As the result of the experiment, it could be seen that in the soybean meal fermented product prepared according to the present invention, the crude protein content was increased, and the content of trypsin inhibitor, beta-conglycinin and indigestible oligosaccharides was significantly decreased, and the lactic acid concentration was increased, compared to the raw soybean meal.

The above result showed that in the fermented soybean meal prepared by the method for preparation of the present invention, the efficacy as fermented soybean meal such as further improved removal of anti-nutritional factors and increase of pepsin digestion rate, etc. was significantly increased, compared to the raw soybean meal, and sufficient component changes and characteristic modification occurred.

In addition, the crude protein content of the fermented soybean meal prepared by using SLB130 strain was higher than the fermented soybean meal prepared by using SLB120 strain, and the content of anti-nutritional factors such as indigestible oligosaccharides, trypsin inhibitors, beta-conglycinin, etc. was significantly reduced. In addition, the lactic acid concentration was increased and thereby the growth of contaminant was inhibited and the cell number of general bacteria was reduced.

For example, in case of Example 5 which used the residual soybean meal as a fermentation raw material, in the fermented soybean meal prepared by SLB130 strain (Example 5-1) compared to the fermented soybean meal using SLB120 strain (Example 5-2), the crude protein content was increased from 56% (w/w) to 60% (w/w) and the representative anti-nutritional factor, trypsin inhibitor was decreased from 1.2 mg/g to 0.8 mg/g in a considerable ratio (30% or more). Also, the content of beta-conglycinin was decreased from 640 ppm to 180 ppm by 50% or more. On the other hand, the indigestible oligosaccharides in soybean meal which played a harmful role by causing flatulence to livestock were significantly reduced from 0.7 mg/g to 0.3 mg/g. In addition, the lactic acid concentration was increased from 3.9% to 5.1%, and thereby the growth of contaminant was inhibited, and thus it was shown that the cell number of general bacteria was lowered.

For example, in case of Example 6 which used the mixed soybean meal as a fermentation raw material, in the fermented soybean meal prepared by SLB130 strain (Example 6-1) compared to the fermented soybean meal using SLB120 strain (Example 6-2), the crude protein content was increased from 51% (w/w) to 53% (w/w) and the representative anti-nutritional factor, trypsin inhibitor was decreased from 2.1 mg/g to 1.0 mg/g in a considerable ratio (50% or more). Also, the content of beta-conglycinin was decreased from 1,500 ppm to 420 ppm by 50% or more. On the other hand, the indigestible oligosaccharides in soybean meal which played a harmful role by causing flatulence to livestock were significantly reduced from 0.12 mg/g to 0.04 mg/g by 60% or more. In addition, the lactic acid concentration was increased from 3.7% to 4.6%, and thereby the growth of contaminant was inhibited, and thus it was shown that the cell number of general bacteria was lowered.

The above result showed that the fermented soybean meal prepared by the new lactic acid bacterium of the present invention, Enterococcus faecium SLB130 strain significantly increased the efficacy as fermented soybean meal such as further improved removal of anti-nutritional factors and increase of pepsin digestion rate, etc. than the fermented soybean meal prepared by the conventional Enterococcus faecium SLB120 strain, and sufficient component changes and characteristic modification occurred.

### EXAMPLE 7

### SDS-PAGE Analysis

### 7-1: Qualitative Analysis

To confirm the degree of increasing the digestion rate as protein in soybean meal was decomposed finely and decomposing beta-conglycinin and trypsin inhibitor, protein among anti-nutritional factors by the fermentation process, lg of each sample of raw soybean meal before fermentation (A), fermented soybean meal of Example 4-2 (B), fermented soybean meal of Example 5-1 (C, D, E) and fermented soybean meal of Example 4-1 (G) was added to 3m1 lysis buffer (urea 7M, thiourea 2M, CAHAPS 4%, DTT 40mM), and after voltexing at a maximum speed for 5 min and then heating at 95° C. for 5 min and centrifuging at 10,000 g for 10 min and then collecting supernatant, the concentration of protein was measured.

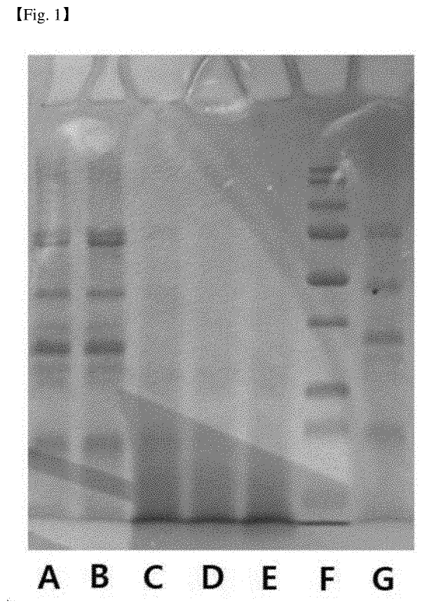

20 ul of the same amount of protein in 12% TGX gel (Biorad) was added into a well and the degree of decomposition of protein was compared by SDS -PAGE. The result was shown in FIG. 1. In FIG. 1, A is the result of SDS-PAGE analysis result of the protein sample of the raw soybean meal before fermentation, and B is the result of SDS-PAGE analysis of the protein sample of the fermented soybean meal (Example 4-2) fermented by using SLB120, and C to E are the results of SDS-PAGE analysis of the protein sample of the fermented soybean meal (Example 5-1) in which the residual soybean meal was fermented by using SLB130, and F is a size marker, and G is the result of SDS-PAGE analysis of the protein sample of the fermented soybean meal (Example 4-1) in which the raw soybean meal was fermented by using SLB130. F of FIG. 1 is a size marker and each line represents the sizes of 250, 150, 100, 75, 50, 37, 25, 20, 15, and 10 kD in order from the top.

As the result, it could be seen that the degree of protein decomposition was increased in case of Example 4-1, and it could be seen that 30 kD or more of protein was mostly decomposed and the peptization of 30 kD or more was the highest in case of Example 5-1. Thus, it could be seen that through the fermentation process, the big size of protein in soybean meal was decomposed finely and the anti-nutritional protein was decomposed and thereby the digestion rate could be increased. In addition, the degree of decomposition in case of fermentation with SBL130 was higher than the case of fermentation with SLB120.

### 7-2: Quantitative Analysis

To investigate the degree of decomposition of protein in soybean meal by the fermentation process, the distribution of molecular weights of protein in the raw soybean meal before fermentation and the soybean meal after fermentation was measured. The result was shown in Table 9. The protein content of the following Table 9 means the weight percent of protein having the corresponding molecular weight range based on 100% by weight of the total protein in fermented soybean meal.

TABLE 9

| | Raw soybean | Exam- | Exam- | Exam- | Exam- |
|---|---|---|---|---|---|
| Molecular weight | meal | ple5-1 | ple6-1 | ple4-1 | ple4-2 |
| Less than 25 kD | 24 | 97 | 51 | 34 | 28 |
| 25 or more to less | 45 | 2 | 40 | 50 | 46 |
| than 50 kD | | | | | |
| 50 kD or more | 31 | 1 | 9 | 16 | 26 |

As a result, the content of high molecule protein of molecular weight 50 kD or more was decreased and the content of protein less than 50 kD was relatively increased through the fermentation process. In particular, it could be confirmed that the content of protein less than 25 kD was particularly increased. In other words, it could be seen that the percentage of low molecule protein was increased than that before fermentation through the fermentation process. In addition, it could be confirmed that the degree of decomposition of protein in case of fermentation with SBL130 was higher than that in case of fermentation with SLB120.

1. A method for preparation of fermented soybean meal, comprising
a step of obtaining soybean meal extract solution and residual soybean meal by extracting raw soybean meal with an extraction solvent, and
a step of performing solid culture of a fermentation raw material comprising one or more kinds selected from the group consisting of raw soybean meal and the residual soybean meal, by using a microorganism fermenting soybean meal.

2. The method for preparation according to claim 1, wherein the microorganism fermenting soybean meal is a facultative anaerobic lactic acid bacterium, and the step of solid culture does not comprise an oxygen aeration process.

3. (canceled)

4. The method for preparation according to claim 1, wherein the extraction solvent is one or more kinds selected from the group consisting of water and alcohols of 1 to 6 carbon atoms, and is used at a weight ratio of 1 to 10 times of the raw soybean meal.

5. The method for preparation according to claim 1, wherein the extraction solvent is 20 to 70° C. of temperature and pH 2 to 8.

6. The method for preparation according to claim 1, wherein the residual soybean meal having a water content of 80 w/w % or less is obtained by extracting the raw soybean meal with an extraction solvent and then separating by a centrifugation process.

7. The method for preparation according to claim 1, wherein the fermentation raw material is a mixture of the residual soybean meal and raw soybean meal, and the crude protein content or anti-nutritional factor content of the fermented soybean meal is controlled by adjusting a mixing ratio.

8. The method for preparation according to claim 7, wherein the anti-nutritional factor is one or more kinds selected from the group consisting of trypsin inhibitor, beta-conglycininin, indigestible oligosaccharide, hemagglutinin (lectin), saponin and tannin.

9. The method for preparation according to claim 1, wherein the fermentation raw material is a mixture in which the raw soybean meal and residual soybean meal are mixed at a weight ratio of 1:10 to 10:1.

10. The method for preparation according to claim 1, wherein the fermentation raw material is obtained from soybean meal, and includes 20 to 48% (w/w) of the crude protein content, and 0.6 to 1.7(w/w %) of the indigestible oligosaccharide content, and
wherein the fermented soybean meal has the crude protein content of higher than 46% (w/w) to lower than 80% (w/w).

11. The method for preparation according to claim 1, comprising
a step of obtaining soybean meal extract solution and residual soybean meal obtained by extracting raw soybean meal with an extraction solvent;
a step of preparing a fermentation raw material in which the crude protein content is 20 to 48% (w/w) and the indigestible oligosaccharide content is 0.6 to 1.7(w/w %), by mixing the raw soybean meal and residual soybean meal, and
a step of performing solid culture of the fermentation raw material, by using a microorganism fermenting soybean meal.

12. The method for preparation according to claim 1, wherein the microorganism fermenting soybean meal is one or more kinds selected from the group consisting of Enterococcus sp. strain, Weissella sp. strain, and Lactobacillus sp. strain.

13. (canceled)

14. (canceled)

15. A soybean meal fermented product, which is obtained by fermenting a fermentation raw material comprising one or more kinds selected from the group consisting of raw soybean meal and residual soybean meal by using a facultative anaerobic soybean meal fermenting microorganism,
wherein the fermentation raw material comprises 20 to 48% (w/w) of the crude protein content and 0.6 to 1.7 (w/w %) of the indigestible oligosaccharide content, and
wherein the soybean meal fermented product comprises 0.0001 to 8 (mg/g) of trypsin inhibitor, 0 to 70,000 (ppm) of beta-conglycinin, 0.0001 to 1.7 (w/w %) of indigestible oligosaccharide, or 46 to 80% (w/w) of crude protein.

16. The soybean meal fermented product according to claim 15, wherein the fermentation raw material comprises one or more kinds selected from the group consisting of raw soybean meal and residual soybean meal which is a solid component obtained by removing soybean meal extract solution in a solvent extract of soybean meal.

17. (canceled)

18. An animal feed composition comprising the fermented product of soybean meal according to claim 15.

19. The feed composition according to claim 18, wherein the animal is one or more kinds selected from the group consisting of pig, cow, chicken, duck, goat, sheep, dog and cat.

20. The feed composition according to claim 18, wherein
(1) the fermented product of soybean meal has the crude protein content of 48 to 53% (w/w), and the animal is adult,
(2) the fermented product of soybean meal has the crude protein content of 50 to 60% (w/w), and the animal is a piglet or chick, or
(3) the fermented product of soybean meal has the crude protein content of 53 to 65% (w/w), and the animal is fish.

21. (canceled)

22. (canceled)

23. The method for preparation according to claim 1, wherein in the fermented soybean meal, the content of protein having a molecular weight of lower than 25 kD is 25 to 99.9% by weight, and the content of protein having a molecular weight of 25 to lower than 50 kD is 0.01 to 60% by weight, and the content of protein having a molecular weight of 50 kD or more is 0.01 to 30% by weight, based on 100% by weight of the total protein in the fermented soybean meal.

24. An Enterococcus faecium strain having the optimum growth temperature of 40 to 45° C.

25. (canceled)

26. The strain according to claim 24, wherein the strain is Enterococcus faecium SLB130 strain deposited with accession number of KCTC13566BP.

27. (canceled)

28. (canceled)

29. (canceled)

30. (canceled)

31. (canceled)

32. (canceled)

33. (canceled)

34. (canceled)

35. (canceled)

36. The method for preparation according to claim 1, further comprising a step of seed culturing that cultures the strain in the soybean meal extract solution obtained by extracting the raw soybean meal with an extraction solvent, before performing the step of fermenting.
